(19) United States
(12) Patent Application Publication
(10) Pub. No.: US 2018/0140648 A1
(43) Pub. Date: May 24, 2018

(54) BACTERIAL POPULATIONS FOR PROMOTING HEALTH

(71) Applicant: Yeda Research and Development Co. Ltd., Rehovot (IL)

(72) Inventors: Eran SEGAL, Ramat-HaSharon (IL); Eran ELINAV, Mazkeret Batia (IL)

(21) Appl. No.: 15/575,827

(22) PCT Filed: May 17, 2016
(86) PCT No.: PCT/IL2016/050520

§ 371 Date: Nov. 21, 2017

Related U.S. Application Data
(60) Provisional application No. 62/256,771, filed on Nov. 18, 2015.
(60) Provisional application No. 62/164,684, filed on May 21, 2015.

(51) Int. Cl. A61K 35/747; A61K 35/745; A61P 3/08

(52) U.S. Cl. CPC A61K 35/747; A61K 2300/00; A61P 3/08; A61K 35/745

(57) ABSTRACT

A method of improving the glucose response in glucose tolerant and intolerant subjects is provided. The method comprises providing to the subject probiotic compositions, or agents which specifically reduce bacterial species.

### FIELD AND BACKGROUND OF THE INVENTION

The present invention, in some embodiments thereof, relates to probiotic and antibiotic compositions for promoting health, in both healthy and diseased subjects.

The prevalence of obesity in adults, children and adolescents has increased rapidly over the past 30 years and continues to rise. Obesity is classically defined based on the percentage of body fat or, more recently, the body mass index (BMI), defined as the ratio of weight (Kg) divided by height (in meters) squared.

Overweight and obesity are associated with increasing the risk of developing many chronic diseases of aging. Such co-morbidities include type 2 diabetes mellitus, hypertension, coronary heart diseases and dyslipidemia, gallstones and cholecystectomy, osteoarthritis, cancer (of the breast, colon, endometrial, prostate, and gallbladder), and sleep apnea. It is recognized that the key to reducing the severity of the diseases is to lose weight effectively. Although about 30 to 40% claim to be trying to lose weight or maintain lost weight, current therapies appear not to be working. Besides dietary manipulation, pharmacological management and in extreme cases, surgery, are sanctioned adjunctive therapies to treat overweight and obese patients. Drugs have side effects, and surgery, although effective, is a drastic measure and reserved for morbidly obese.

Background art includes Ivey et al., European Journal of Clinical Nutrition 68, 447-452 (April 2014).

### SUMMARY OF THE INVENTION

According to an aspect of some embodiments of the present invention, there is provided a method of preventing diabetes or pre-diabetes in a subject comprising administering to the subject at least one bacteria of a phylum, class, order, family, genus or species of a bacteria which is categorized as beneficial according to Table 3, thereby preventing diabetes or prediabetes in the subject.

According to an aspect of some embodiments of the present invention, there is provided a method of preventing diabetes or pre-diabetes in a subject comprising administering to the subject an agent which specifically reduces at least one bacteria of a phylum, class, order, family, genus or species of a bacteria which is categorized as non-beneficial according to Table 3, thereby preventing diabetes or prediabetes in the subject.

According to an aspect of some embodiments of the present invention, there is provided a method of preventing diabetes or pre-diabetes in a subject comprising administering to the subject at least one bacteria having a Kegg pathway or module which is categorized as beneficial according to Table 3, thereby preventing diabetes or prediabetes in the subject.

According to an aspect of some embodiments of the present invention, there is provided a method of preventing diabetes or pre-diabetes in a subject comprising administering to the subject an agent which specifically reduces at least one bacteria having a Kegg pathway or module which is categorized as non-beneficial according to Table 3, thereby preventing diabetes or prediabetes in the subject.

According to an aspect of some embodiments of the present invention, there is provided a probiotic composition, comprising at least two bacteria of a phylum, class, order, family, genus or species of a bacteria which is categorized as beneficial according to Table 3.

According to an aspect of some embodiments of the present invention, there is provided a probiotic composition, comprising at least two bacteria of a phylum, class, order, family, genus or species of a bacteria having a Kegg pathway or module which is categorized as beneficial according to Table 3.

According to an aspect of some embodiments of the present invention, there is provided a pharmaceutical composition comprising as the active agent an agent which specifically reduces the number of bacteria having a Kegg pathway or module which is categorized as non-beneficial according to Table 3.

According to an aspect of some embodiments of the present invention, there is provided a pharmaceutical composition comprising as the active agent an agent which specifically reduces the number of bacteria of a phylum, class, order, family, genus or species of bacteria which is categorized as non-beneficial according to Table 3. According to an aspect of some embodiments of the present invention, there is provided a method of preventing diabetes or pre-diabetes in a subject comprising administering to the subject at least one bacteria of a phylum, class, order, family, genus or species of a bacteria which is categorized as beneficial according to Table 4, thereby preventing diabetes or prediabetes in the subject.

According to an aspect of some embodiments of the present invention, there is provided a method of preventing diabetes or pre-diabetes in a subject comprising administering to the subject an agent which specifically reduces at least one bacteria of a phylum, class, order, family, genus or species of a bacteria which is categorized as non-beneficial according to Table 4, thereby preventing diabetes or prediabetes in the subject.

According to an aspect of some embodiments of the present invention, there is provided a method of preventing diabetes or pre-diabetes in a subject comprising administering to the subject at least one bacteria having a Kegg pathway or module which is categorized as beneficial according to Table 4, thereby preventing diabetes or prediabetes in the subject.

According to an aspect of some embodiments of the present invention, there is provided a method of preventing diabetes or pre-diabetes in a subject comprising administering to the subject an agent which specifically reduces at least one bacteria having a Kegg pathway or module which is categorized as non-beneficial according to Table 4, thereby preventing diabetes or prediabetes in the subject.

According to an aspect of some embodiments of the present invention, there is provided a probiotic composition, comprising at least two bacteria of a phylum, class, order, family, genus or species of a bacteria which is categorized as beneficial according to Table 4.

According to an aspect of some embodiments of the present invention, there is provided a probiotic composition, comprising at least two bacteria of a phylum, class, order, family, genus or species of a bacteria having a Kegg pathway or module which is categorized as beneficial according to Table 4.

According to an aspect of some embodiments of the present invention, there is provided a pharmaceutical composition comprising as the active agent an agent which specifically reduces the number of bacteria having a Kegg pathway or module which is categorized as non-beneficial according to Table 4.

According to an aspect of some embodiments of the present invention, there is provided a pharmaceutical composition comprising as the active agent an agent which specifically reduces the number of bacteria of a phylum, class, order, family, genus or species of bacteria which is categorized as non-beneficial according to Table 4.

According to an aspect of some embodiments of the present invention, there is provided a method of preventing diabetes or pre-diabetes in a subject comprising administering to the subject at least one bacteria of a phylum, class, order, family, genus or species of a bacteria which is categorized as beneficial according to Table 5, thereby preventing diabetes or prediabetes in the subject.

According to an aspect of some embodiments of the present invention, there is provided a method of preventing diabetes or pre-diabetes in a subject comprising administering to the subject an agent which specifically reduces at least one bacteria of a phylum, class, order, family, genus or species of a bacteria which is categorized as non-beneficial according to Table 5, thereby preventing diabetes or prediabetes in the subject.

According to an aspect of some embodiments of the present invention, there is provided a probiotic composition, comprising at least two bacteria of a phylum, class, order, family, genus or species of a bacteria which is categorized as beneficial according to Table 5.

According to an aspect of some embodiments of the present invention, there is provided a pharmaceutical composition comprising as the active agent an agent which specifically reduces the number of bacteria of a phylum, class, order, family, genus or species of bacteria which is categorized as non-beneficial according to Table 5.

According to an aspect of some embodiments of the present invention, there is provided a method of improving the glucose response in a glucose intolerant subject comprising providing to the subject a probiotic composition comprising at least one bacteria species selected from the group consisting of Coprococcus sp. ART55/1 draft, vButyrate-producing bacterium SSC/2, Roseburia intestinalis XB6B4 draft, Eubacterium siraeum V10Sc8a draft, Veillonella parvula DSM 2008 chromosome, Ruminococcus sp. SR1/5 draft, Ruminococcus bromii L2-63 draft, Bacteroides thetaiotaomicron VPI-5482 chromosome, Faecalibacterium prausnitzii L2-6, Bifidobacterium adolescentis ATCC 15703 chromosome, Ruminococcus obeum A2-162 draft, Bacteroides xylanisolvens XB1A draft, Treponema succinifaciens DSM 2489 chromosome, Bacteroides vulgatus ATCC 8482 chromosome, Klebsiella pneumoniae subsp. pneumoniae HS11286 chromosome, Eubacterium siraeum 70/3 draft, Bifidobacterium bifidum BGN4 chromosome, Methanobrevibacter smithii ATCC 35061 chromosome, Eubacterium eligens ATCC 27750 chromosome, Eubacterium rectale M104/1 draft, Megamonas hypermegale ART12/1 draft, Lactobacillus ruminis ATCC 27782 chromosome, Escherichia coli SE15, Streptococcus pyogenes MGAS2096 chromosome, Bifidobacterium longum subsp. longum F8 draft, Klebsiella pneumoniae JM45, Escherichia coli str. ‘clone D i2’ chromosome, Klebsiella oxytoca KCTC 1686 chromosome, Raoultella ornithinolytica B6, Methylocella silvestris, Roseiflexus castenholzii and Streptococcus macedonicus, wherein the probiotic composition does not comprise more than 50 species of bacteria, thereby improving the glucose response in a glucose intolerant subject.

According to an aspect of some embodiments of the present invention, there is provided a method of improving the glucose response in a glucose intolerant subject comprising providing to the subject an agent which specifically reduces the number of bacteria of a species selected from the group consisting of Streptococcus thermophilus ND03 chromosome, Bifidobacterium longum subsp. infantis 157F chromosome, Alistipes finegoldii DSM 17242 chromosome, Streptococcus salivarius CCHSS3, Shigella sonnei 53G, Lactococcus lactis subsp. lactis 111403 chromosome, Bifidobacterium breve UCC2003, Shigella flexneri 2002017 chromosome, Enterococcus sp. 7L76 draft, Klebsiella oxytoca E718 chromosome, Enterobacter cloacae subsp. cloacae ATCC 13047 chromosome, Streptococcus oralis Uo5, Shigella sonnei Ss046 chromosome, Escherichia coli JJ1886, Streptococcus thermophilus LMG 18311 chromosome, Escherichia coli APEC 01 chromosome, Gardnerella vaginalis 409-05 chromosome, Escherichia coli CFT073 chromosome, Escherichia coli ED1a chromosome, Enterobacter cloacae EcWSU1 chromosome, Enterobacter asburiae LF7a chromosome, Enterococcus faecalis str. Symbioflor 1, Granulicella mallensis, Campylobacter jejuni and Arthrospira platensis, thereby improving the glucose response in a glucose intolerant subject.

According to an aspect of some embodiments of the present invention, there is provided a method of maintaining the glucose response in a glucose tolerant subject comprising providing to the subject an agent which specifically reduces the number of bacteria of a species selected from the group consisting of Streptococcus salivarius CCHSS3, Shigella sonnei 53G, Akkermansia muciniphila ATCC BAA-835 chromosome, Klebsiella pneumoniae subsp. pneumoniae MGH 78578 chromosome, Bifidobacterium longum DJO10A chromosome, Enterobacter cloacae subsp. cloacae NCTC 9394 draft, Escherichia coli str. K-12 substr. DH10B chromosome, Streptococcus thermophilus CNRZ1066 chromosome, Faecalibacterium prausnitzii SL3/3 draft, Escherichia coli O7:K1 str. CE10 chromosome, Methylocella silvestris, Roseiflexus castenholzii and Streptococcus macedonicus, thereby maintaining the glucose response in a glucose tolerant subject.

According to an aspect of some embodiments of the present invention, there is provided a method of maintaining the glucose response in a glucose tolerant subject comprising providing to the subject a probiotic composition comprising at least one bacterial subspecies selected from the group consisting of Streptococcus thermophilus LMD-9, Streptococcus thermophilus ND03 chromosome, Bifidobacterium longum subsp. infantis 157F chromosome, Bifidobacterium animalis subsp. lactis V9 chromosome, Faecalibacterium prausnitzii L2-6, Escherichia coli JJ1886, Lactococcus garvieae ATCC 49156, Streptococcus thermophilus MN-ZLW-002 chromosome, Lactobacillus acidophilus La-14, Granulicella mallensis, Campylobacter jejuni and Arthrospira platensis thereby maintaining the glucose response in a glucose tolerant subject, wherein the probiotic composition does not comprise more than 50 species of bacteria.

According to an aspect of some embodiments of the present invention, there is provided a method of improving the health of a subject comprising administering to the subject a bacterial composition wherein the majority of the bacteria of the composition are of the genus selected from the group consisting of Advenella, Vibrio and Brachyspira.

According to an aspect of some embodiments of the present invention, there is provided a method of improving the health of a subject comprising administering to the subject an agent which specifically reduces the number of bacteria being of the genus selected from the group consisting of Spiroplasma, Ferrimonas, Nautilia, Cupriavidus and Helicobacter.

According to an aspect of some embodiments of the present invention, there is provided a method of improving the health of a subject comprising administering to the subject an agent which specifically reduces the number of bacteria being of the phylum selected from the group consisting of proteobacteria and verrucomicrobia.

According to an aspect of some embodiments of the present invention, there is provided a probiotic composition, wherein a majority of the bacteria of the composition are microbes of the Advenella, Vibrio and/or Brachyspira genus, the composition being formulated for rectal or oral administration.

According to an aspect of some embodiments of the present invention, there is provided a probiotic composition, comprising at least two microbe species selected from the group consisting of Coprococcus sp. ART55/1 draft, vButyrate-producing bacterium SSC/2, Roseburia intestinalis XB6B4 draft, Eubacterium siraeum V10Sc8a draft, Veillonella parvula DSM 2008 chromosome, Ruminococcus sp. SR1/5 draft, Ruminococcus bromii L2-63 draft, Bacteroides thetaiotaomicron VPI-5482 chromosome, Faecalibacterium prausnitzii L2-6, Bifidobacterium adolescentis ATCC 15703 chromosome, Ruminococcus obeum A2-162 draft, Bacteroides xylanisolvens XB1A draft, Treponema succinifaciens DSM 2489 chromosome, Bacteroides vulgatus ATCC 8482 chromosome, Klebsiella pneumoniae subsp. pneumoniae HS11286 chromosome, Eubacterium siraeum 70/3 draft, Bifidobacterium bifidum BGN4 chromosome, Methanobrevibacter smithii ATCC 35061 chromosome, Eubacterium eligens ATCC 27750 chromosome, Eubacterium rectale M104/1 draft, Megamonas hypermegale ART12/1 draft, Lactobacillus ruminis ATCC 27782 chromosome, Escherichia coli SE15, Streptococcus pyogenes MGAS2096 chromosome, Bifidobacterium longum subsp. longum F8 draft, Klebsiella pneumoniae JM45, Escherichia coli str. ‘clone D i2’ chromosome, Klebsiella oxytoca KCTC 1686 chromosome, Raoultella ornithinolytica B6, Granulicella mallensis, Campylobacter jejuni and Arthrospira platensis, wherein the composition does not comprise more than 50 species of bacteria, the composition being formulated for rectal or oral administration.

According to an aspect of some embodiments of the present invention, there is provided a probiotic composition, comprising at least two bacteria species selected from the group consisting of Streptococcus thermophilus LMD-9, Streptococcus thermophilus ND03 chromosome, Bifidobacterium longum subsp. infantis 157F chromosome, Bifidobacterium animalis subsp. lactis V9 chromosome, Faecalibacterium prausnitzii L2-6, Escherichia coli JJ1886, Lactococcus garvieae ATCC 49156, Streptococcus thermophilus MN-ZLW-002 chromosome, Lactobacillus acidophilus La-14, Granulicella mallensis, Campylobacter jejuni and Arthrospira platensis, wherein the probiotic composition does not comprise more than 50 species of bacteria, the composition being formulated for rectal or oral administration.

According to an aspect of some embodiments of the present invention, there is provided a pharmaceutical composition comprising as the active agent an agent which specifically reduces the number of bacteria of a species selected from the group consisting of species selected from the group consisting of Streptococcus thermophilus ND03 chromosome, Bifidobacterium longum subsp. infantis 157F chromosome, Alistipes finegoldii DSM 17242 chromosome, Streptococcus salivarius CCHSS3, Shigella sonnei 53G, Lactococcus lactis subsp. lactis 111403 chromosome, Bifidobacterium breve UCC2003, Shigella flexneri 2002017 chromosome, Enterococcus sp. 7L76 draft, Klebsiella oxytoca E718 chromosome, Enterobacter cloacae subsp. cloacae ATCC 13047 chromosome, Streptococcus oralis Uo5, Shigella sonnei Ss046 chromosome, Escherichia coli JJ1886, Streptococcus thermophilus LMG 18311 chromosome, Escherichia coli APEC 01 chromosome, Gardnerella vaginalis 409-05 chromosome, Escherichia coli CFT073 chromosome, Escherichia coli ED1a chromosome, Enterobacter cloacae EcWSU1 chromosome, Enterobacter asburiae LF7a chromosome, Enterococcus faecalis str. Symbioflor 1, Granulicella mallensis, Campylobacter jejuni and Arthrospira platensis, and a pharmaceutically acceptable carrier.

According to an aspect of some embodiments of the present invention, there is provided a pharmaceutical composition comprising as the active agent an agent which specifically reduces the number of bacteria of a species selected from the group consisting of Streptococcus salivarius CCHSS3, Shigella sonnei 53G, Akkermansia muciniphila ATCC BAA-835 chromosome, Klebsiella pneumoniae subsp. pneumoniae MGH 78578 chromosome, Bifidobacterium longum DJO10A chromosome, Enterobacter cloacae subsp. cloacae NCTC 9394 draft, Escherichia coli str. K-12 substr. DH10B chromosome, Streptococcus thermophilus CNRZ1066 chromosome, Faecalibacterium prausnitzii SL3/3 draft, Escherichia coli O7:K1 str. CE10 chromosome, Methylocella silvestris, Roseiflexus castenholzii and Streptococcus macedonicus, and a pharmaceutically acceptable carrier.

According to an aspect of some embodiments of the present invention, there is provided a pharmaceutical composition comprising as the active agent an agent which specifically reduces the number of bacteria being of the genus selected from the group consisting of Spiroplasma, Ferrimonas, Nautilia, Cupriavidus and Helicobacter, and a pharmaceutically acceptable carrier.

According to an aspect of some embodiments of the present invention, there is provided a pharmaceutical composition comprising as the active agent an agent which specifically reduces the number of bacteria being of the phylum selected from the group consisting of proteobacteria and verrucomicrobia, and a pharmaceutically acceptable carrier.

According to some embodiments of the invention, the glucose intolerant subject is a diabetic subject or a prediabetic subject.

According to some embodiments of the invention, the subject is a healthy subject.

According to some embodiments of the invention, the subject has a metabolic disorder.

According to some embodiments of the invention, the metabolic disorder is diabetes or pre-diabetes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph illustrating that the average glycemic response in the good week is lower compared to the bad week. Average iAUCmed level of 16 participants in the good (green) and bad (red) weeks. iAUCmed is the incremental area under the curve (AUC) above the median glucose level 15 minutes before the meal was consumed. The iAUCmed level of a participant is the average iAUCmed of all its breakfasts, lunches and dinners. In the x-axis, IG signifies an impaired glucose participant and H signifies a healthy participant. The first number after the symbol IG/H in the brackets is the average wakeup glucose level of 6 days of experiment and the second number in the brackets is the HbA1C at the beginning of the experiment).

FIGS. 2A-B are diagrams illustrating that Bacteroides thehaitaomicron VPI-5482 changes its abundance during different diets. The order of the weeks displayed is mix week followed by the bad week and the good week is displayed last although the order of the good and bad weeks were randomly chosen for participants. FIG. 2A: Participants who chronologically ate the bad diet following the good diet. FIG. 2B: Participants who chronologically are the good diet following the bad diet. Legend PD signifies impaired glucose participants and N signifies healthy participants.

FIGS. 3A-B are graphs illustrating the glucose response of participants meals (y-axis) as a function of the amount of carbohydrates (in grams) content of the meals for four individuals.

FIG. 4 is a heat map illustrating the abundance of different phylum of bacteria associated with blood glucose levels and carbohydrate sensitivity.

FIG. 5 is a heat map illustrating the abundance of different genus of bacteria associated with blood glucose levels and carbohydrate sensitivity.

FIG. 6 is a heat map illustrating the abundance of different species of bacteria associated with blood glucose levels and carbohydrate sensitivity.

FIG. 7 is a heatmap (subset) of statistically significant associations (P<0.05, FDR corrected) between participants' standardized meals PPGRs and participants' clinical and microbiome data.

FIGS. 8A-G illustrate factors underlying the prediction of postprandial glycemic responses (PPGRs). (A) Partial dependence plot (PDP) showing the marginal contribution of the meal's carbohydrate content to the predicted PPGR (y-axis, arbitrary units) at each amount of meal carbohydrates (x-axis). Red and green indicate above and below zero contributions, respectively (number indicate meals). Boxplots (bottom) indicate the carbohydrates content at which different percentiles (10, 25, 50, 75, and 90) of the distribution of all meals across the cohort are located. See PDP legend. (B) Histogram of the slope (computed per participant) of a linear regression between the carbohydrate content and the PPGR of all meals. Also shown is an example of one participant with a low slope and another with a high slope. (C) Meal fat/carbohydrate ratio PDP. (D) Histogram of the difference (computed per participant) between the Pearson R correlation of two linear regression models, one between the PPGR and the meal carbohydrate content and another when adding fat and carbohydrate*fat content. Also shown is an example of the carbohydrate and fat content of all meals of one participant with a relatively low R difference (carb alone correlates well with PPGR) and another with a relatively high difference (meals with high fat content have lower PPGRs). Dot color and size correspond to the meal's PPGR. (E) Additional PDPs. (F) Microbiome PDPs. The number of participants in which the microbiome feature was not detected is indicated (left, n.d.). Boxplots (box, IQR; whiskers 10-90 percentiles) based only on detected values. (G) Heatmap of statistically significant correlations (Pearson) between microbiome features termed beneficial (green) or non-beneficial (red) and several risk factors and glucose parameters.

FIG. 9 are partial dependency plots (PDPs, as in FIGS. 8A-G), for additional features underlying the prediction of postprandial glycemic responses.

FIGS. 10A-E illustrate that dietary interventions induce consistent alterations to the gut microbiota composition. (A) Top: Continuous glucose measurements of a participant from the expert arm for both the ‘bad’ diet (left) and ‘good’ diet (right) week. Bottom: Fold change between the relative abundance (RA) of taxa in each day of the ‘bad’ (left) or ‘good’ (right) weeks and days 0-3 of the same week. Shown are only taxa that exhibit statistically significant changes with respect to a null hypothesis of no change derived from changes in the first profiling week (no intervention) of all participants. (B) As in (A) for a participant from the predictor arm. See also Table 5 for changes in all participants. (C) Heatmap of taxa with opposite trends of change in RA between ‘good’ and ‘bad’ intervention weeks that was consistent across participant and statistically significant (Mann-Whitney U-test between changes in the ‘good’ and ‘bad’ weeks, P<0.05, FDR corrected). Left and right column blocks shows bacteria increasing and decreasing in their RA following the ‘good’ diet, respectively, and conversely for the ‘bad’ diet. Colored entries represent the (log) fold change between the RA of a taxon (x-axis) between days 4-7 and 0-3 within each participant (y-axis). (D) For Bifidobacterium adolescentis, which decreased significantly following the ‘good’ diet interventions (see panel C), shown is the average and standard deviation of the (log) fold change of all participants in each day of the ‘good’ (top) diet week relative to days 0-3 of the ‘good’ week. Same for the ‘bad’ diet week (bottom) in which B. adolescentis increases significantly (see panel C). Grey lines show fold changes (log) in individual participants. (E) As in (D), for Roseburia inulinivorans.

### DESCRIPTION OF SPECIFIC EMBODIMENTS OF THE INVENTION

The present invention, in some embodiments thereof, relates probiotic and antibiotic compositions for promoting health in both healthy and diseased subjects.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The gut microbiome is in constant flux, continuously changing its microbial composition in response to external stimuli such as food intake, antibiotic intake and disease. As such, the phylogenetic compositions of microbiomes vary from one individual to another. Such differences have been associated with diseases such as colon cancer and inflammatory bowel disease, susceptibility to obesity, the severity of autism spectrum disorders, and differences in responses to medical treatments.

It is known that the bacterial content of the gut microbiome changes according to the type of foods that are ingested. The present inventors analyzed the gut microbiome in pre-diabetic and healthy subjects that were exposed to foods that were pre-selected to promote a high or low glucose response. They found that certain bacteria were enriched in the microbiome of subjects who responded to the food with a low glucose response, whilst other bacteria were depleted in the microbiome of subjects who responded to the food with a low glucose response as compared to the microbiome of subjects who responded to the food with a high glucose response.

The present inventors propose to take advantage of the knowledge of the bacterial composition of the microbiomes following ingestion of each of these diets to formulate pro- or anti-biotic compositions to promote health and well-being.

Whilst further reducing the present invention to practice, the present inventors profiled overall blood glucose response as well as sensitivity to intake of carbohydrates in healthy and prediabetic subjects. The present inventors analyzed the microbiome composition in groups of subjects classified as having a high or low blood glucose response as well as in subjects classified as being more or less sensitive to carbohydrates as measured by blood glucose levels. Analysis of the bacterial content of the microbiome content in each of these groups allowed the present inventors to propose additional bacterial populations which correlate with the low blood glucose response and/or sensitivity to carbohydrates.

The presently disclosed compositions can be used to reduce the risk of developing metabolic diseases such as diabetes or prediabetes, or to delay the onset of the disease. The present compositions can be used to reduce the risk of developing associated complications and/or delay the onset of such complications.

Thus, according to a first aspect of the present invention there is provided a method of preventing diabetes or pre-diabetes in a subject comprising administering to the subject at least one bacteria of a phylum, class, order, family, genus or species of a bacteria which is categorized as beneficial according to any one of Tables 3-5, thereby preventing diabetes or prediabetes in the subject.

According to still another aspect of the present invention, there is provided a method of preventing diabetes or pre-diabetes in a subject comprising administering to the subject at least one bacteria having a Kegg pathway or module which is categorized as beneficial according to any one of Tables 3 or 4, thereby preventing diabetes or prediabetes in the subject.

As used herein, the term “probiotic” refers to any microbial type that is associated with health benefits in a host organism and/or reduction of risk and/or symptoms of a disease, disorder, condition, or event in a host organism. In some embodiments, probiotics are formulated in a food product, functional food or nutraceutical. In some embodiments, probiotics are types of bacteria.

Diabetic conditions include, for example, type 1 diabetes, type 2 diabetes, gestational diabetes, pre-diabetes, slow onset autoimmune diabetes type 1 (LADA), hyperglycemia, and metabolic syndrome. The diabetes may be overt, diagnosed diabetes, e.g., type 2 diabetes, or a pre-diabetic condition.

Diabetes mellitus (generally referred to herein as “diabetes”) is a disease that is characterized by impaired glucose regulation. Diabetes is a chronic disease that occurs when the pancreas fails to produce enough insulin or when the body cannot effectively use the insulin that is produced, resulting in an increased concentration of glucose in the blood (hyperglycemia). Diabetes may be classified as type 1 diabetes (insulin-dependent, juvenile, or childhood-onset diabetes), type 2 diabetes (non-insulin-dependent or adult-onset diabetes), LADA diabetes (late autoimmune diabetes of adulthood) or gestational diabetes. Additionally, intermediate conditions such as impaired glucose tolerance and impaired fasting glycemia are recognized as conditions that indicate a high risk of progressing to type 2 diabetes.

In type 1 diabetes, insulin production is absent due to autoimmune destruction of pancreatic beta-cells. There are several markers of this autoimmune destruction, detectable in body fluids and tissues, including islet cell autoantibodies, insulin autoantibodies, glutamic acid decarboxylase autoantibodies, and tyrosine phosphatase ICA512/IA-2 autoantibodies. In type 2 diabetes, comprising 90% of diabetics worldwide, insulin secretion may be inadequate, but peripheral insulin resistance is believed to be the primary defect. Type 2 diabetes is commonly, although not always, associated with obesity, a cause of insulin resistance.

Type 2 diabetes is often preceded by pre-diabetes, in which blood glucose levels are higher than normal but not yet high enough to be diagnosed as diabetes.

The term “pre-diabetes,” as used herein, is interchangeable with the terms “Impaired Glucose Tolerance” or “Impaired Fasting Glucose,” which are terms that refer to tests used to measure blood glucose levels.

Chronic hyperglycemia in diabetes is associated with multiple, primarily vascular complications affecting microvasculature and/or macrovasculature. These long-term complications include retinopathy (leading to focal blurring, retinal detachment, and partial or total loss of vision), nephropathy (leading to renal failure), neuropathy (leading to pain, numbness, and loss of sensation in limbs, and potentially resulting in foot ulceration and/or amputation), cardiomyopathy (leading to heart failure), and increased risk of infection. Type 2, or noninsulin-dependent diabetes mellitus (NIDDM), is associated with resistance of glucose-utilizing tissues like adipose tissue, muscle, and liver, to the physiological actions of insulin. Chronically elevated blood glucose associated with NIDDM can lead to debilitating complications including nephropathy, often necessitating dialysis or renal transplant; peripheral neuropathy; retinopathy leading to blindness; ulceration and necrosis of the lower limbs, leading to amputation; fatty liver disease, which may progress to cirrhosis; and susceptibility to coronary artery disease and myocardial infarction.

The probiotic composition of this aspect of the present invention may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or all of the bacterial phylum, class, order, family, genus or species categorized as being beneficial in Tables 3, Table 4 and/or Table 5.

According to one embodiment, the probiotic composition does not comprise more than 2 bacterial species, 5 bacterial species, 10 bacterial species, 15 bacterial species, 20 bacterial species, 25 bacterial species, 30 bacterial species, 35 bacterial species, 40 bacterial species, 45 bacterial species, 50 bacterial species, 55 bacterial species, 60 bacterial species, 65 bacterial species, 70 bacterial species, 75 bacterial species, 80 bacterial species, 85 bacterial species, 90 bacterial species, 95 bacterial species, 100 bacterial species, 150 bacterial species, 200 bacterial species, 250 bacterial species or 300 bacterial species.

According to other embodiments, the probiotic composition does not comprise more than 2 bacterial species, 5 bacterial species, 10 bacterial species, 15 bacterial species, 20 bacterial species, 25 bacterial species, 30 bacterial species, 35 bacterial species, 40 bacterial species, 45 bacterial species, 50 bacterial species, 55 bacterial species, 60 bacterial species, 65 bacterial species, 70 bacterial species, 75 bacterial species, 80 bacterial species, 85 bacterial species, 90 bacterial species, 95 bacterial species, 100 bacterial species, 150 bacterial species, 200 bacterial species, 250 bacterial species or 300 bacterial species which are categorized as being non-beneficial according to Table 3, Table 4 and/or Table 5.

According to another embodiment, the probiotic composition does not comprise more than 2 bacterial phylum, 5 bacterial phylum or more than 10 bacterial phylum.

According to another embodiment, the probiotic composition does not comprise more than 2 bacterial phylum, 5 bacterial phylum or more than 10 bacterial phylum which are categorized as being non-beneficial according to Table 3, Table 4 and/or Table 5.

According to another embodiment, the probiotic composition does not comprise more than 2 bacterial class, 5 bacterial class or more than 10 bacterial class.

According to another embodiment, the probiotic composition does not comprise more than 2 bacterial class, 5 bacterial class or more than 10 bacterial class which are categorized as being non-beneficial according to Tables 3, Table 4 and/or Table 5.

According to another embodiment, the probiotic composition does not comprise more than 2 bacterial order, 5 bacterial order or more than 10 bacterial order.

According to another embodiment, the probiotic composition does not comprise more than 2 bacterial order, 5 bacterial order or more than 10 bacterial order which are categorized as being non-beneficial according to Table 3, Table 4, and/or Table 5.

According to another embodiment, the probiotic composition does not comprise more than 2 bacterial genus, 5 bacterial genus or more than 10 bacterial genus.

According to another embodiment, the probiotic composition does not comprise more than 2 bacterial genus, 5 bacterial genus or more than 10 bacterial genus which are categorized as being non-beneficial according to Table 3, Table 4 and/or Table 5.

According to another embodiment, the probiotic composition does not comprise more than 2 bacterial families, 5 bacterial families or more than 10 bacterial families.

According to another embodiment, the probiotic composition does not comprise more than 2 bacterial families, 5 bacterial families or more than 10 bacterial families which are categorized as being non-beneficial according to Table 3, Table 4 and/or Table 5.

According to still another embodiment, at least 20%, 30%, 40%, 50%, 60%, 70%, 80%, 90% of the bacteria in the probiotic composition have a KEGG pathway or module as listed in Table 3 and/or Table 4.

It will be appreciated in the case of discrepancy or inconsistencies amongst bacterial populations between Tables 3-5, the data in Table 5 should prevail.

According to still another aspect of the present invention, there is provided a method of improving the glucose response in a glucose intolerant subject comprising providing to the subject a probiotic composition comprising at least one bacteria species selected from the group consisting of Coprococcus sp. ART55/1 draft, vButyrate-producing bacterium SSC/2, Roseburia intestinalis XB6B4 draft, Eubacterium siraeum V10Sc8a draft, Veillonella parvula DSM 2008 chromosome, Ruminococcus sp. SR1/5 draft, Ruminococcus bromii L2-63 draft, Bacteroides thetaiotaomicron VPI-5482 chromosome, Faecalibacterium prausnitzii L2-6, Bifidobacterium adolescentis ATCC 15703 chromosome, Ruminococcus obeum A2-162 draft, Bacteroides xylanisolvens XB1A draft, Treponema succinifaciens DSM 2489 chromosome, Bacteroides vulgatus ATCC 8482 chromosome, Klebsiella pneumoniae subsp. pneumoniae HS11286 chromosome, Eubacterium siraeum 70/3 draft, Bifidobacterium bifidum BGN4 chromosome, Methanobrevibacter smithii ATCC 35061 chromosome, Eubacterium eligens ATCC 27750 chromosome, Eubacterium rectale M104/1 draft, Megamonas hypermegale ART12/1 draft, Lactobacillus ruminis ATCC 27782 chromosome, Escherichia coli SE15, Streptococcus pyogenes MGAS2096 chromosome, Bifidobacterium longum subsp. longum F8 draft, Klebsiella pneumoniae JM45, Escherichia coli str. ‘clone D i2’ chromosome, Klebsiella oxytoca KCTC 1686 chromosome, Raoultella ornithinolytica B6, Methylocella silvestris, Roseiflexus castenholzii and Streptococcus macedonicus, wherein the probiotic composition does not comprise more than 50 species of bacteria, thereby improving the glucose response in a glucose intolerant subject.

It will be appreciated in the case of discrepancy or inconsistencies amongst bacterial populations between those disclosed above and those disclosed in Tables 3-5, the data in Tables 3-5 should prevail, and more preferably the data in Table 5 should prevail.

As used herein, the term “glucose intolerant subject” refers to a subject that has a threshold fasting plasma glucose (FPG) greater than 100 mg/dl and/or a threshold 2-hour oral glucose tolerance test (OGTT) glucose level greater than 140 mg/dl.

The term “species” as used herein refers to both a species and subspecies.

According to one embodiment, the subject has metabolic condition such as diabetes or pre-diabetes.

The probiotic composition of this aspect of the present invention may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or all of the bacterial species listed.

According to one embodiment, the probiotic composition does not comprise more than 2 bacterial species, 5 bacterial species, 10 bacterial species, 15 bacterial species, 20 bacterial species, 25 bacterial species, 30 bacterial species, 35 bacterial species, 40 bacterial species, 45 bacterial species, 50 bacterial species, 55 bacterial species, 60 bacterial species, 65 bacterial species, 70 bacterial species, 75 bacterial species, 80 bacterial species, 85 bacterial species, 90 bacterial species, 95 bacterial species, 100 bacterial species, 150 bacterial species, 200 bacterial species, 250 bacterial species or 300 bacterial species.

According to another aspect of the present invention, there is provided a method of maintaining the glucose response in a glucose tolerant subject (or preventing diabetes) comprising providing to the subject a probiotic composition comprising at least one bacterial species selected from the group consisting of Streptococcus thermophilus LMD-9, Streptococcus thermophilus ND03 chromosome, Bifidobacterium longum subsp. infantis 157F chromosome, Bifidobacterium animalis subsp. lactis V9 chromosome, Faecalibacterium prausnitzii L2-6, Escherichia coli JJ1886, Lactococcus garvieae ATCC 49156, Streptococcus thermophilus MN-ZLW-002 chromosome, Lactobacillus acidophilus La-14, Granulicella mallensis, Campylobacter jejuni and Arthrospira platensis thereby maintaining the glucose response in a glucose tolerant subject, wherein the probiotic composition does not comprise more than 50 species of bacteria.

The term “glucose tolerant” subject refers to a subject that has a threshold fasting plasma glucose (FPG) lower than 100 mg/dl and/or a threshold 2-hour oral glucose tolerance test (OGTT) glucose level lower than 140 mg/dl.

The probiotic composition of this aspect of the present invention may comprise 1, 2, 3, 4, 5, 6, 7, 8, 9, 10, 11 or all of the bacterial species listed.

According to one embodiment, the probiotic composition of this aspect of the present invention does not comprise more than 2 bacterial species, 5 bacterial species, 10 bacterial species, 15 bacterial species, 20 bacterial species, 25 bacterial species, 30 bacterial species, 35 bacterial species, 40 bacterial species, 45 bacterial species, 50 bacterial species, 55 bacterial species, 60 bacterial species, 65 bacterial species, 70 bacterial species, 75 bacterial species, 80 bacterial species, 85 bacterial species, 90 bacterial species, 95 bacterial species, 100 bacterial species, 150 bacterial species, 200 bacterial species, 250 bacterial species or 300 bacterial species.

According to still another aspect of the present invention, there is provided a method of improving the health of a subject comprising administering to the subject a bacterial composition wherein the majority of the bacteria of the composition are of the genus selected from the group consisting of Advenella, Vibrio and Brachyspira.

According to this aspect of the present invention, the subject may be healthy or have a disease. The subject may be glucose tolerant or glucose intolerant.

According to a particular embodiment, the subject has a disease such as diabetes, hyperlipidemia (also referred to as hyperlipoproteinemia, or hyperlipidaemia), a liver disease or disorder including hepatitis, cirrhosis, non-alcoholic steatohepatitis (NASH) (also known as non-alcoholic fatty liver disease-NAFLD), hepatotoxicity and chronic liver disease.

The compositions of this aspect of the present invention may comprise 1, 2, 3, 4, 5, 6, 7, 8, 9, 10, 20, 30, 40, 50 or more species belonging to the Advenella, Vibrio and/or Brachyspira genus.

In one embodiment, the composition may consist entirely of bacteria belonging to the Advenella genus, the Vibrio genus and/or Brachyspira genus.

According to still another embodiment, the microbial composition of any of the aspects of the present invention is devoid (or comprises only trace quantities) of fecal material (e.g., fiber).

The probiotic bacteria may be in any suitable form, for example in a powdered dry form. In addition, the probiotic microorganism may have undergone processing in order for it to increase its survival. For example, the microorganism may be coated or encapsulated in a polysaccharide, fat, starch, protein or in a sugar matrix. Standard encapsulation techniques known in the art can be used. For example, techniques discussed in U.S. Pat. No. 6,190,591, which is hereby incorporated by reference in its entirety, may be used.

According to a particular embodiment, the probiotic microorganism composition is formulated in a food product, functional food or nutraceutical.

In some embodiments, a food product, functional food or nutraceutical is or comprises a dairy product. In some embodiments, a dairy product is or comprises a yogurt product. In some embodiments, a dairy product is or comprises a milk product.

In some embodiments, a dairy product is or comprises a cheese product. In some embodiments, a food product, functional food or nutraceutical is or comprises a juice or other product derived from fruit. In some embodiments, a food product, functional food or nutraceutical is or comprises a product derived from vegetables. In some embodiments, a food product, functional food or nutraceutical is or comprises a grain product, including but not limited to cereal, crackers, bread, and/or oatmeal. In some embodiments, a food product, functional food or nutraceutical is or comprises a rice product. In some embodiments, a food product, functional food or nutraceutical is or comprises a meat product.

Prior to administration, the subject may be pretreated with an agent which reduces the number of naturally occurring microbes in the microbiome (e.g. by antibiotic treatment). According to a particular embodiment, the treatment significantly eliminates the naturally occurring gut microflora by at least 20%, 30% 40%, 50%, 60%, 70%, 80% or even 90%.

As well as probiotic compositions, the present inventors also propose the use of agents that specifically reduce the numbers of particular bacteria.

Thus, according to yet another aspect of the present invention there is provided a method of preventing diabetes or pre-diabetes in a subject comprising administering to the subject an agent which specifically reduces at least one bacteria of a phylum, class, order, family, genus or species of a bacteria which is categorized as non-beneficial according to any one of Tables 3-5, thereby preventing diabetes or prediabetes in the subject.

According to still another aspect of the present invention there is provided a method of preventing diabetes or pre-diabetes in a subject comprising administering to the subject an agent which specifically reduces at least one bacteria having a Kegg pathway or module which is categorized as non-beneficial according to any one of Tables 3 or 4, thereby preventing diabetes or prediabetes in the subject.

According to yet another aspect of the present invention, there is provided a method of improving the glucose response in a glucose intolerant subject comprising providing to the subject an agent which specifically reduces the number of bacteria of a species selected from the group consisting of Streptococcus thermophilus ND03 chromosome, Bifidobacterium longum subsp. infantis 157F chromosome, Alistipes finegoldii DSM 17242 chromosome, Streptococcus salivarius CCHSS3, Shigella sonnei 53G, Lactococcus lactis subsp. lactis 111403 chromosome, Bifidobacterium breve UCC2003, Shigella flexneri 2002017 chromosome, Enterococcus sp. 7L76 draft, Klebsiella oxytoca E718 chromosome, Enterobacter cloacae subsp. cloacae ATCC 13047 chromosome, Streptococcus oralis Uo5, Shigella sonnei Ss046 chromosome, Escherichia coli JJ1886, Streptococcus thermophilus LMG 18311 chromosome, Escherichia coli APEC 01 chromosome, Gardnerella vaginalis 409-05 chromosome, Escherichia coli CFT073 chromosome, Escherichia coli ED1a chromosome, Enterobacter cloacae EcWSU1 chromosome, Enterobacter asburiae LF7a chromosome, Enterococcus faecalis str. Symbioflor 1, Granulicella mallensis, Campylobacter jejuni and Arthrospira platensis, thereby improving the glucose response in a glucose intolerant subject.

According to another aspect of the present invention, there is provided a method of maintaining the glucose response in a glucose tolerant subject comprising providing to the subject an agent which specifically reduces the number of bacteria of a species selected from the group consisting of Streptococcus salivarius CCHSS3, Shigella sonnei 53G, Akkermansia muciniphila ATCC BAA-835 chromosome, Klebsiella pneumoniae subsp. pneumoniae MGH 78578 chromosome, Bifidobacterium longum DJ010A chromosome, Enterobacter cloacae subsp. cloacae NCTC 9394 draft, Escherichia coli str. K-12 substr. DH10B chromosome, Streptococcus thermophilus CNRZ1066 chromosome, Faecalibacterium prausnitzii SL3/3 draft, Escherichia coli O7:K1 str. CE10 chromosome, Methylocella silvestris, Roseiflexus castenholzii and Streptococcus macedonicus, thereby maintaining the glucose response in a glucose tolerant subject.

According to still another aspect, there is provided a method of improving the health of a subject comprising administering to the subject an agent which specifically reduces the number of bacteria being of the genus selected from the group consisting of Spiroplasma, Ferrimonas, Nautilia, Cupriavidus and Helicobacter.

According to still another aspect there is provided a method of improving the health of a subject comprising administering to the subject an agent which specifically reduces the number of bacteria being of the phylum selected from the group consisting of proteobacteria and verrucomicrobia.

As used herein, the phrase “specifically reduce” refers to an ability to reduce by least 2 fold a bacteria as compared to another bacteria of the microbiome of the subject. According to a particular embodiment, the agent reduces the particular bacteria by at least 5 fold, 10 fold or more as compared to the other bacteria of the microbiome.

As used herein, the term “microbiome” refers to the totality of microbes (bacteria, fungae, protists), their genetic elements (genomes) in a defined environment.

The microbiome may be a gut microbiome, an oral microbiome, a bronchial microbiome, a skin microbiome or a vaginal microbiome.

According to a particular embodiment, the microbiome is a gut microbiome (i.e. intestinal microbiome).

According to one embodiment, the agent reduces the species of bacteria by at least 2 fold as compared to a different species of bacteria that belongs to the same genus present in the microbiome.

According to a particular embodiment the agent reduces the species of bacteria by at least 5 fold, 10 fold or more as compared to another species of bacteria that belongs to the same genus present in the microbiome.

According to one embodiment, the agent reduces the genus of bacteria by at least 2 fold as compared to a different genus of bacteria that belongs to the same family present in the microbiome.

According to a particular embodiment, the agent reduces the genus of bacteria by at least 5 fold, 10 fold or more as compared to another genus of bacteria that belongs to the same family present in the microbiome.

According to one embodiment, the agent reduces the phylum of bacteria by at least 2 fold as compared to a different phylum of bacteria that belongs to the same kingdom present in the microbiome.

According to a particular embodiment, the agent reduces the phylum of bacteria by at least 5 fold, 10 fold or more as compared to another phylum of bacteria that belongs to the same kingdom present in the microbiome.

Agents that specifically reduce a particular bacterial species are known in the art and include polynucleotide silencing agents.

Preferably, the polynucleotide silencing agent of this aspect of the present invention targets a sequence that encodes an essential genes (i.e., compatible with life) in the bacteria. The sequence which is targeted should be specific to the particular bacteria species/phylum or genus that it is desired to down-regulate. Such genes include ribosomal RNA genes (16S and 23S), ribosomal protein genes, tRNA-synthetases, as well as additional genes shown to be essential such as dnaB, fabI, folA, gyrB, murA, pytH, metG, and tufA(B) NC_009641 for Staphylococcus aureus subsp. aureus str. Newman and NC_003485 for Streptococcus pyogenes MGAS8232 (DeVito et al., Nature Biotechnology 20, 478-483 (2002)).

According to an embodiment of the invention, the polynucleotide silencing agent is specific to a target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

miRNA and miRNA Mimics—

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al., 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—

Antisense is a single stranded RNA designed to prevent or inhibit expression of a gene by specifically hybridizing to its mRNA. Downregulation of a bacteria can be effected using an antisense polynucleotide capable of specifically hybridizing with an mRNA transcript encoding a bacterial gene.

Design of antisense molecules which can be used to efficiently downregulate a particular sequence specific to a bacteria must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al., Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating an essential gene in a bacteria is a ribozyme molecule capable of specifically cleaving an mRNA transcript encoding the gene. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Another agent capable of downregulating an essential bacterial gene is a RNA-guided endonuclease technology e.g. CRISPR system.

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

An additional method of regulating the expression of an essential bacterial gene is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al., Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al., J Biol Chem, 2001; 276:28991-98), and the sequence- and target specific downregulation of expression of the Ets2 transcription factor, important in prostate cancer etiology (Carbone, et al., Nucl Acid Res. 2003; 31:833-43), and the pro-inflammatory ICAM-1 gene (Besch et al., J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003017068 and 2003096980 to Froehler et al., and 200 0128218 and 20020123476 to Emanuele et al., and U.S. Pat. No. 5,721,138 to Lawn.

In some embodiments, administering comprises any means of administering an effective (e.g., therapeutically effective) or otherwise desirable amount of a composition to an individual. In some embodiments, administering a composition comprises administration by any route, including for example parenteral and non-parenteral routes of administration. Parenteral routes include, e.g., intraarterial, intracerebroventricular, intracranial, intramuscular, intraperitoneal, intrapleural, intraportal, intraspinal, intrathecal, intravenous, subcutaneous, or other routes of injection. Non-parenteral routes include, e.g., buccal, nasal, ocular, oral, pulmonary, rectal, transdermal, or vaginal. Administration may also be by continuous infusion, local administration, sustained release from implants (gels, membranes or the like), and/or intravenous injection.

In some embodiments, a composition is administered in an amount and/or according to a dosing regimen that is correlated with a particular desired outcome (e.g., down-regulation of a particular bacterial species).

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of diabetes and/or level of risk of diabetes, etc., or combinations thereof). Such doses or amounts can be determined by those of ordinary skill. In some embodiments, an appropriate dose or amount is determined in accordance with standard clinical techniques. Alternatively or additionally, in some embodiments, an appropriate dose or amount is determined through use of one or more in vitro or in vivo assays to help identify desirable or optimal dosage ranges or amounts to be administered.

In some particular embodiments, appropriate doses or amounts to be administered may be extrapolated from dose-response curves derived from in vitro or animal model test systems. The effective dose or amount to be administered for a particular individual can be varied (e.g., increased or decreased) over time, depending on the needs of the individual. In some embodiments, where bacteria are administered, an appropriate dosage comprises at least about 100, 200, 300, 400, 500, 600, 700, 800, 900, 1000 or more bacterial cells. In some embodiments, the present invention encompasses the recognition that greater benefit may be achieved by providing numbers of bacterial cells greater than about 1000 or more (e.g., than about 1500, 2000, 2500, 3000, 35000, 4000, 4500, 5000, 5500, 6000, 7000, 8000, 9000, 10,000, 15,000, 20,000, 25,000, 30,000, 40,000, 50,000, 75,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 1×10⁸, 1×10⁹, 1×10¹⁰, 1×10¹¹, 1×10¹², 1×10¹³ or more bacteria.

According to another embodiment, the agent which is capable of specifically reducing a particular bacteria is an antibiotic.

As used herein, the term “antibiotic agent” refers to a group of chemical substances, isolated from natural sources or derived from antibiotic agents isolated from natural sources, having a capacity to inhibit growth of, or to destroy bacteria, and other microorganisms, used chiefly in treatment of infectious diseases. Examples of antibiotic agents include, but are not limited to; Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

According to a particular embodiment, the antibiotic is a non-absorbable antibiotic.

It is expected that during the life of a patent maturing from this application many relevant antibiotics will be developed and the scope of the term antibiotic is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Effect of Diet on Bacterial Populations

Materials and Methods

16 impaired glycemic response and healthy participants engaged in a three week experiment of diet intervention. The first week was a profiling week, from which two personalized test diets were computed: (1) one full week of a personalized diet predicted to have “good” (low) postprandial blood glucose responses; and (2) one full week of a personalized diet predicted to have “bad” (high) postprandial blood glucose responses. The present inventors evaluated whether indeed the personalized diet of the “good” week elicited lower blood glucose responses as compared to the personalized diet given on the “bad” week.

Before the experiment, a dietitian planned a personal tailored diet for 6 days as follows: each participant decided how many meals and calories he or she eats in a day. All meals in the 6 days were different and in every day the same number of meals and calories were consumed with a gap of at least 3 hours between meals. The content of the meals was decided by the participant to match their taste and regular diet. For example, a participant may choose to eat 5 meal categories a day as following: a 300 calorie breakfast, 200 calorie brunch, 500 calorie launch, 200 calorie snack and 800 calorie dinner. The participant decides on 6 different options for each meal category (5 meal categories in the example: breakfasts, brunch, launch, snack and dinner) with the help of the dietitian to ensure that all breakfasts are isocaloric with a maximum deviation of 10%.

The experiment began with taking a blood sample and anthropometric measurements from the participant, connecting the participant to a continuous glucose monitor and starting the 6 day diet, while logging all eaten meals during the time of the study. On the 7^{th} day of the experiment, the participant performed a standard (50 g) oral glucose tolerance test after which he ate normally throughout that day. The first week which is referred to as the “mix week” exposed the participant to a variety of foods which afterwards determined which meals were relatively “good” and “bad” i.e. which meals resulted in low and high glucose response respectively. The glucose blood levels were monitored using a continuous glucose monitor (Medtronic iPro2) with a high 5 minute temporal resolution. The glucose rise and glucose incremental area under the curve (AUC) was measured for each meal. The meals from low to high response were selected where the best and worst two meals of every meal category were selected and marked as good meals and bad meals.

After the good and bad meals were selected, the participants continued with the additional two weeks of the experiment, which were the test weeks. The “good week” comprised only of good meals and “bad week” comprised only of meals predicted to elicit “bad” (high) blood glucose responses. A week comprised 6 days of diet and one day of 50 grams glucose tolerance test as described above. The order of the weeks was randomly chosen and neither participant nor dietitian were exposed to the order of the weeks. After three weeks, the glucose level between weeks was compared.

To date, 16 individuals completed the experiment out of which 10 had an impaired glycemic response and 6 were healthy.

Bacterial Samples:

Bacterial samples were 100 bp paired-end sequenced with at least 1 million reads per sample using Illumina NextSeq 500 sequencer. Reads were mapped to full genomes NCBI's non-redundant database using GEM mapper and bacterial relative abundance were then computed. Bacteria that appeared in relative abundance of at least 0.1% of any sample were monitored.

Results

“Good” and “bad” meals were correctly categorized: It was found that the vast majority of the meals tested in the two test weeks showed a glucose response in accord with the predictions (low/high).

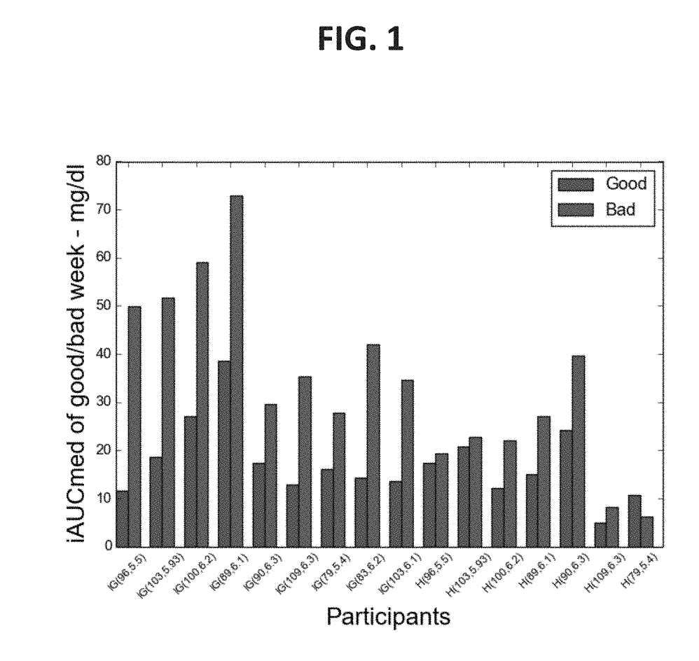

A significant improvement in the average AUC following a meal in the “good” week compared to the “bad” week was observed. This result holds for both healthy and impaired glucose tolerance individuals where in the latter group the differences between the “good” and “bad” week were greater (FIG. 1).

80 bacteria were identified that significantly changed their relative abundance either after the ‘good’ week or after the ‘bad’ week. These bacteria represent potential targets for intervention as follows: beneficial bacteria are those that significantly increase in abundance during the good week or that significantly decrease during the bad week; detrimental bacteria are those that significantly increase in abundance during the bad week or that significantly decrease during the good week. The bacteria that changed in prediabetic subjects are summarized in Table 1 herein below.

TABLE 1

| Bacteria | Prediabetic Direction | | Prediabetic P-Value | |
|---|---|---|---|---|
| name | good week | bad week | good week | bad week |
| Coprococcus sp. | 0.54 | −0.71 | 0.09 | 0.04 |
| ART55/1 draft | | | | |
| Butyrate-producing | 0.79 | −0.79 | 0.02 | 0.02 |
| bacterium SSC/2 | | | | |
| Streptococcus | 0.44, −0.24, | 1.45, 0.97, 0.1 | 0.13, 0.27, 0.00007 | 0.0001, 0.008, 0.39 |
| thermophilus LMD-9 | 1.54 | | | |
| Streptococcus | −1.41, 0.41, −2.24 | 0.27, 1.85, −0.16 | 2.7e−04, | 0.24, 3.0e−6, 0.28 |
| thermophilus | | | 0.14, 2.09e−08 | |
| ND03 chromosome | | | | |
| Bifidobacterium | −0.22, −2.66, −0.5 | 1.94, 2.58, 2.52 | 0.22, 3.78e−11, | 9.8e−07, 1.24e−10, |
| longum subsp. | | | 4.54e−2 | 3.22e−10 |
| infantis 157F | | | | |
| chromosome | | | | |
| Alistipes finegoldii | −0.77 | 0.1 | 0.02 | 0.39 |
| DSM 17242 | | | | |
| chromosome | | | | |
| Roseburia intestinalis | 0.47 | −0.84 | 0.11 | 0.017 |
| XB6B4 draft | | | | |
| Streptococcus | −0.51, −0.43 | 0.84, 1.66 | 0.1, 0.13 | 0.01, 0.00002 |
| salivarius CCHSS3 | | | | |
| Eubacterium | −0.29, −1.72, | 0.65, 1.6, −2.28 | 0.16, 1.2e−5, | 1.5e−02, 4.43e−05, |
| rectale ATCC 33656 | 1.66 | | 2.4e−5, | 1.2e−08 |
| Eubacterium siraeum | 0.74 | −0.26 | 0.034 | 0.25 |
| V10Sc8a draft | | | | |
| Veillonella parvula | 0.07 | −0.95 | 0.43 | 0.009 |
| DSM 2008 chromosome | | | | |
| Shigella sonnei 53G | −0.91 | 0.15 | 0.01 | 0.34 |
| Bifidobacterium | 2.04, −1.79, | 0, −0.08, 1.64 | 0, 6.0e−6, 0.44 | 0.5, 0.42, 0.00002 |
| animalis subsp. | 0.02 | | | |
| lactis V9 chromosome | | | | |
| Lactococcus lactis | −0.69 | 0.03 | 0.04 | 0.46 |
| subsp. | | | | |
| lactis Il1403 | | | | |
| chromosome | | | | |
| Streptococcus salivarius | −0.3 | −0.78 | 0.23 | 0.02 |
| JIM8777 | | | | |
| Ruminococcus sp. | 0.67 | −0.73 | 0.01 | 0.006 |
| SR1/5 | | | | |
| draft | | | | |
| Ruminococcus bromii | 0.65 | −0.53 | 0.01 | 0.03 |
| L2-63 | | | | |
| draft | | | | |
| Bacteroides | 0.59 | −0.71 | 0.02 | 0.008 |
| thetaiotaomicron | | | | |
| VPI-5482 chromosome | | | | |
| Acidaminococcus | −0.28 | −0.82 | 0.24 | 0.02 |
| intestini | | | | |
| RyC-MR95 | | | | |
| chromosome | | | | |
| Faecalibacterium | 1.57 | 0 | 0.000056 | 0.5 |
| prausnitzii | | | | |
| L2-6 | | | | |
| Akkermansia | 1.002, −0.54, | −0.02, 1.29, 1.22 | 0.007, 0.002, 0.0001 | 0.47, 8.0e−6, 0.001 |
| muciniphila | −1.47 | | | |
| ATCC BAA-835 | | | | |
| chromosome | | | | |
| Bifidobacterium | 0.95 | −1.23 | 0.009 | 0.001 |
| adolescentis | | | | |
| ATCC 15703 | | | | |
| chromosome | | | | |
| Ruminococcus obeum | 0.56 | −0.43 | 0.03 | 0.07 |
| A2-162 | | | | |
| draft | | | | |
| Eubacterium rectale | 1.94, 0.56, −0.14 | −0.22, 1.55, 2.74 | 0.000001, 0.08, 0.31 | 0.28, 7.02e−05, |
| DSM 17629 | | | | 9.64e−12 |
| draft | | | | |
| Bacteroides | 0.71, 0.72 | −0.85, −0.2 | 0.03, 0.03 | 0.01, 0.3 |
| xylanisolvens XB1A | | | | |
| draft | | | | |
| Treponema | 0.7 | −0.42 | 0.04 | 0.14 |
| succinifaciens DSM | | | | |
| 2489 chromosome | | | | |
| Bifidobacterium breve | −0.77 | 0.39 | 0.02 | 0.17 |
| UCC2003 | | | | |
| Bacteroides vulgatus | 0.32 | −0.08 | 0.04 | 0.33 |
| ATCC 8482 | | | | |
| chromosome | | | | |
| Klebsiella pneumoniae | 0 | −0.72 | 0.5 | 0.03 |
| subsp. | | | | |
| pneumoniae HS11286 | | | | |
| chromosome | | | | |
| Shigella flexneri | −0.49 | 0.8 | 0.115 | 0.02 |
| 2002017 | | | | |
| chromosome | | | | |
| Eubacterium siraeum | 0.56, 1.15, 0.62 | −0.29, −0.42, | 0.03, 0.002, 0.059 | 0.16, 0.07, 0.04 |
| 70/3 | | 0.69 | | |
| draft | | | | |
| Bifidobacterium bifidum | 2.26 | −2.57 | 1.47e−08 | 1.39e−10 |
| BGN4 | | | | |
| chromosome | | | | |
| Methanobrevibacter | −0.19 | −0.72 | 0.26 | 0.007 |
| smithii | | | | |
| ATCC 35061 | | | | |
| chromosome | | | | |
| Enterococcus sp. 7L76 | −1.86 | 2.38 | 0.000003 | 2.76e−09 |
| draft | | | | |
| Eubacterium eligens | 2.16, 0.74 | −0.89, −1.19 | 5.8e−08, 3.37e−02 | 0.01, 0.001 |
| ATCC | | | | |
| 27750 chromosome | | | | |
| Eubacterium rectale | 1.97, 2.04 | −0.7, −0.24 | 0.000001, 0 | 0.03, 0.2 |
| M104/1 | | | | |
| draft | | | | |
| Klebsiella oxytoca E718 | −0.67 | 1.48 | 0.04 | 0.0001 |
| chromosome | | | | |
| Enterobacter cloacae | −1.13 | 1.27 | 0.002 | 0.0009 |
| subsp. | | | | |
| cloacae ATCC 13047 | | | | |
| chromosome | | | | |
| Streptococcus oralis Uo5 | −0.41 | 0.89 | 0.15 | 0.01 |
| Megamonas | −0.07, 0.39 | −1.87, −0.66 | 0.42, 0.09 | 2.0e−6, 0.01 |
| hypermegale | | | | |
| ART12/1 draft | | | | |
| Lactobacillus ruminis | 0.89 | −0.4 | 0.01 | 0.16 |
| ATCC 27782 | | | | |
| chromosome | | | | |
| Roseburia intestinalis | 0, 0.16 | 2.54, −2.46 | 0.5, 0.29 | 2.53e−10, 8.58e−10 |
| M50/1 draft | | | | |
| Shigella sonnei Ss046 | −1.15 | 1.32 | 0.002 | 0.0005 |
| chromosome | | | | |
| Escherichia coli SE15 | 0 | −1.79 | 0.5 | 6.0e−6 |
| Streptococcus pyogenes | 0.67 | −0.54 | 0.04 | 0.09 |
| MGAS2096 | | | | |
| chromosome | | | | |
| Escherichia coli JJ1886 | 0, −2.14 | −0.72, 0 | 0.5, 8.18e−8 | 0.03, 0.5 |
| Bifidobacterium longum | 0, 2.45, −0.42 | −1.53, −0.76, −1.85 | 0.5, 9.69e−10, | 8.8e−5, 0.02, 3.0e−6 |
| subsp. longum F8 draft | | | 0.14 | |
| Escherichia coli | 0.84 | 0 | 0.01 | 0.5 |
| UMN026 | | | | |
| chromosome | | | | |
| Bifidobacterium bifidum | −2.85, 0.62 | 0.69, −1.66 | 1.43e−12, 6.44e−02 | 0.01, 0.00002 |
| PRL2010 chromosome | | | | |
| Lactococcus lactis | 0.89, 0.007 | 0, 1.26 | 0.01, 0.49 | 0.5, 0.001 |
| subsp. | | | | |
| lactis CV56 | | | | |
| chromosome | | | | |
| Bifidobacterium | −0.08 | −0.78 | 0.42 | 0.02 |
| animalis | | | | |
| subsp. lactis CNCM I- | | | | |
| 2494 | | | | |
| chromosome | | | | |
| Streptococcus | −1.32 | 1.05 | 0.0006 | 0.004 |
| thermophilus | | | | |
| LMG 18311 | | | | |
| chromosome | | | | |
| Bifidobacterium | 0.94 | 0.21 | 0.01 | 0.29 |
| animalis subsp. | | | | |
| lactis B1-04 chromosome | | | | |
| Streptococcus | −0.86, 1.24 | 0.64, 0 | 0.01, 0.001 | 0.05, 0.5 |
| constellatus | | | | |
| subsp. pharyngis C818 | | | | |
| Escherichia coli APEC | −0.73 | 1.2 | 0.03 | 0.001 |
| O1 chromosome | | | | |
| Bifidobacterium longum | 0, −1.44 | −2.09, 1.24 | 0.5, 0.0002 | 0, 0.001 |
| subsp. | | | | |
| longum BBMN68 | | | | |
| chromosome | | | | |
| Gardnerella vaginalis | −0.88 | 1.13 | 0.01 | 0.002 |
| 409-05 | | | | |
| chromosome | | | | |
| Lactobacillus gasseri | −0.8 | −0.32 | 0.02 | 0.2 |
| ATCC | | | | |
| 33323 chromosome | | | | |
| Klebsiella pneumoniae | 1.34 | 0.6 | 0.0005 | 0.06 |
| JM45 | | | | |
| Lactobacillus salivarius | −0.13 | −0.7 | 0.37 | 0.04 |
| CECT | | | | |
| 5713 chromosome | | | | |
| Escherichia coli str. | 1.32 | −2.13 | 0.0006 | 8.69e−08 |
| clone D i2’ | | | | |
| chromosome | | | | |
| Escherichia coli CFT073 | −0.78 | 0.55 | 0.02 | 0.08 |
| chromosome | | | | |
| Escherichia coli ED1a | −2.34 | 2.59 | 5.12e−09 | 1.06e−10 |
| chromosome | | | | |
| Klebsiella oxytoca | 1.52 | 0 | 0.000098 | 0.5 |
| KCTC 1686 | | | | |
| chromosome | | | | |
| Enterobacter cloacae | 0.34 | 1.14 | 0.19 | 0.002 |
| EcWSU1 | | | | |
| chromosome | | | | |
| Enterobacter asburiae | −1.37 | 1.72 | 0.0003 | 0.000012 |
| LF7a | | | | |
| chromosome | | | | |
| Raoultella | 1.51 | 0.12 | 0.0001 | 0.37 |
| ornithinolytica B6 | | | | |
| Enterococcus faecalis | −0.81 | 1.32 | 0.02 | 0.0006 |
| str. | | | | |
| Symbioflor 1 | | | | |

The bacteria that changed in healthy subjects are summarized in Table 2 herein below.

TABLE 2

| Bacteria | Prediabetic Direction | | Prediabetic P-Value | |
|---|---|---|---|---|
| name | good week | bad week | good week | bad week |
| Streptococcus | 0.28 | −1.24 | 0.24 | 0.001 |
| thermophilus LMD-9 | | | | |
| Streptococcus | 0, 0 | −2.34, −1.13 | 0.5, 0.5 | 4.55e−09, 2.8e−03 |
| thermophilus | | | | |
| ND03 chromosome | | | | |
| Bifidobacterium | 2.47 | −0.36 | 6.85e−10 | 0.18 |
| longum subsp. | | | | |
| infantis 157F | | | | |
| chromosome | | | | |
| Streptococcus | −0.50 | 1.05 | 0.1 | 0.004 |
| salivarius CCHSS3 | | | | |
| Eubacterium | 0.75, 0.31, 2.63 | 0.21, 2.73, 0 | 3.23e−02, | 0.29, 1.05e−11, |
| rectale ATCC 33656 | | | 0.14, 5.87e−11 | 0.5 |
| Shigella sonnei 53G | −0.07 | 0.68 | 0.42 | 0.04 |
| Bifidobacterium | 0.57 | −2.24 | 0.07 | 2.13e−08 |
| animalis subsp. | | | | |
| lactis V9 chromosome | | | | |
| Faecalibacterium prausnitzii | 0 | −0.98 | 0.5 | 0.007 |
| L2-6 | | | | |
| Akkermansia muciniphila | −0.82 | 1.14 | 0.01 | 0.002 |
| ATCC BAA-835 | | | | |
| chromosome | | | | |
| Bifidobacterium | −0.73 | 0.49 | 0.03 | 0.1 |
| adolescentis | | | | |
| ATCC 15703 chromosome | | | | |
| Enterococcus sp. 7L76 draft | 0.67 | 0 | 0.04 | 0.5 |
| Klebsiella oxytoca E718 | 1.12, −1.28 | 0, 0.97 | 0.003, 0.0008 | 0.5, 0.008 |
| chromosome | | | | |
| Roseburia intestinalis | 0.673 | 0.45 | 0.012 | 0.13 |
| M50/1 draft | | | | |
| Escherichia coli JJ1886 | 1.21 | −1.02 | 0.001 | 0.005 |
| Klebsiella pneumoniae | −0.69 | 0.5 | 0.04 | 0.1 |
| subsp. | | | | |
| pneumoniae MGH 78578 | | | | |
| chromosome | | | | |
| Bifidobacterium longum | −0.87 | 0.375 | 0.01 | 0.1 |
| DJO10A chromosome | | | | |
| Lactococcus garvieae | 1.40 | 0 | 0.0002 | 0.5 |
| ATCC 49156 | | | | |
| Enterobacter cloacae subsp. | −1.19 | 0.9 | 0.001 | 0.01 |
| cloacae NCTC 9394 draft | | | | |
| Escherichia coli str. K-12 | −2.06 | 1.17 | 0 | 0.001 |
| substr. | | | | |
| DH10B chromosome | | | | |
| Streptococcus thermophilus | −0.52 | 1.3 | 0.09 | 0.0006 |
| CNRZ1066 chromosome | | | | |
| Lactococcus lactis subsp. | 0.4 | 0.78 | 0.16 | 0.02 |
| cremoris A76 chromosome | | | | |
| Streptococcus thermophilus | 0.99 | 0 | 0.007 | 0.5 |
| MN-ZLW-002 chromosome | | | | |
| Lactobacillus acidophilus | 0.18 | −0.93 | 0.32 | 0.01 |
| La-14 | | | | |
| Faecalibacterium prausnitzii | −0.15 | 2.34 | 0.3 | 4.74e−09 |
| SL3/3 draft | | | | |
| Escherichia coli O7:K1 str. | 0 | 0.9 | 0.5 | 0.01 |
| CE10 chromosome | | | | |

In the second and third column of Tables 1 and 2 the change in abundance (log_10) during the good and bad week are provided, respectively. The fourth and fifth columns represent the p-value of these abundance changes.

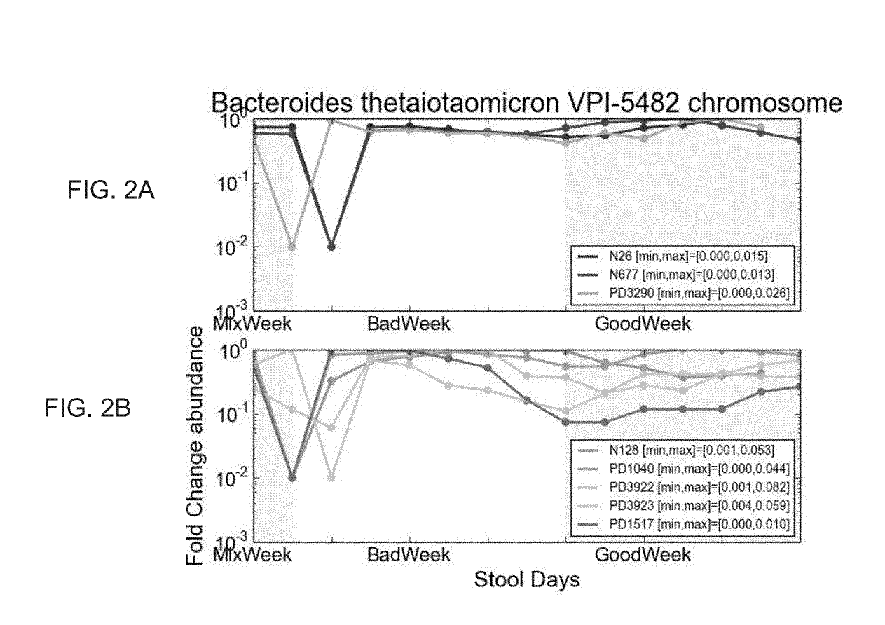

Of the 80 bacteria that we found to significantly change during the diet intervention weeks, most were previously shown to be associated with bacteria-host relationships. For example, bacteria Bacteroides thetaiotaomicron which is considered as a beneficial and important bacteria in hydrolyzing otherwise indigestible dietary polysaccharides, decreases its relative abundance in the bad week and increases in the good week in individuals with impaired glucose responses (FIGS. 2A-B).

### Example 2

### Bacteria Significantly Associated with High Blood Glucose Response to Food

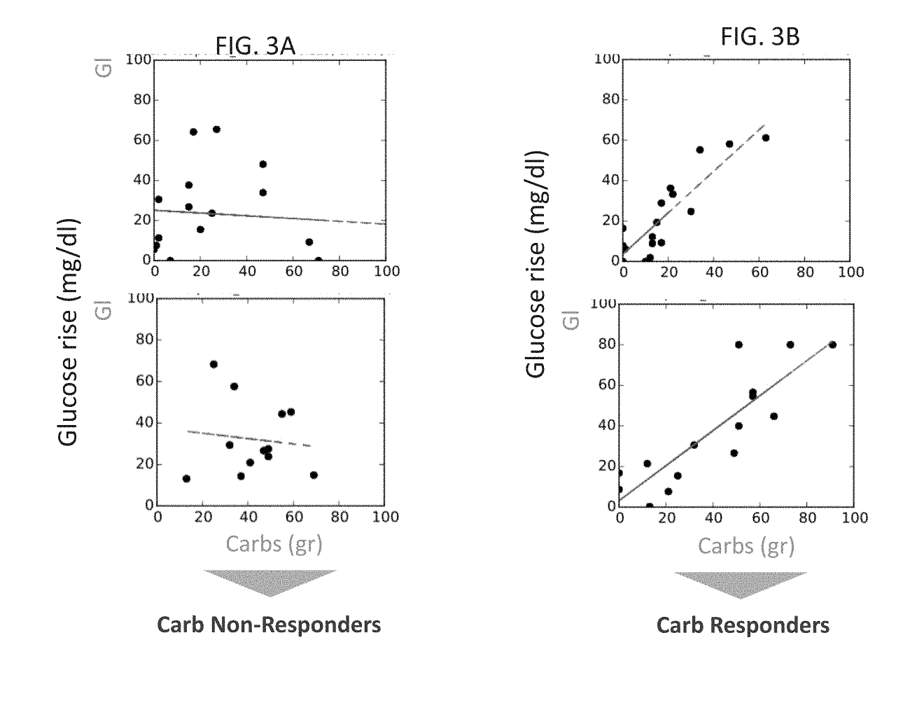

182 participants were profiled comparing their overall blood glucose response (“Median glucose”) as well as their sensitivity to intake of carbohydrates (“Carb-Response”). Median glucose was computed as the median level of blood glucose during the entire week in which the participant was connected to a continuous glucose monitor. Carb response was the linear slope of the graph linking the glucose response of the participant to all meals consumed during the week to the amount of carbohydrates (in grams) in the meal. High slopes indicate that high sensitivity in the glucose responses of the individual to the amount of carbs in the meal and low slopes indicate a low sensitivity to carb intake (FIGS. 3A-B).

For each of these features (median glucose and carb response), the association between the feature and multiple different microbiome signatures was computed.

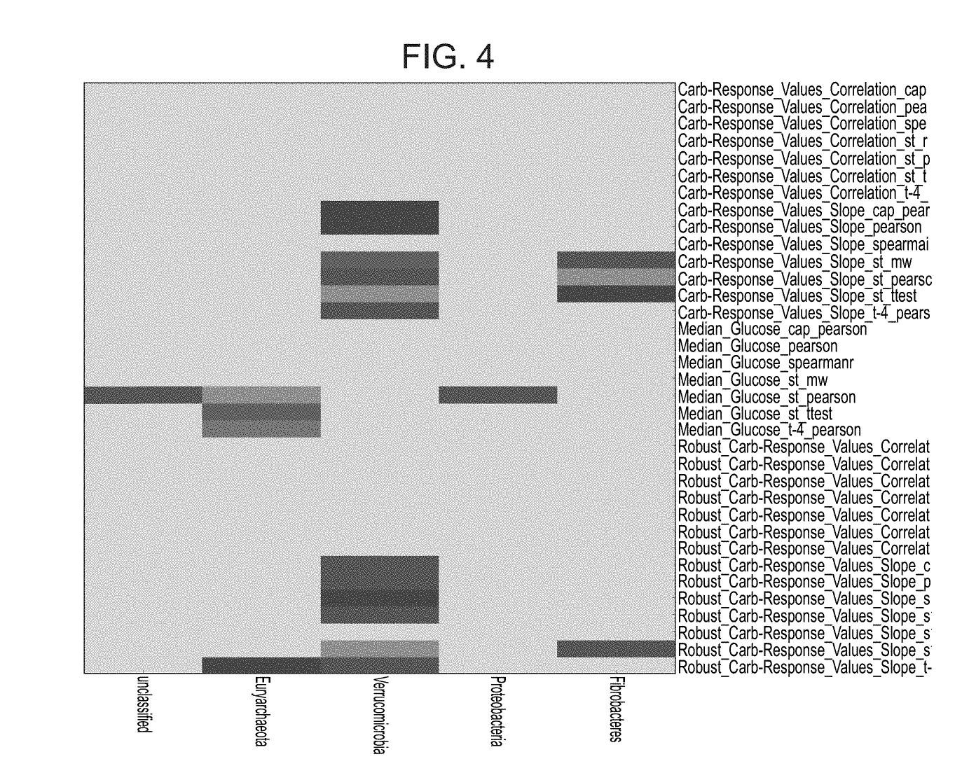

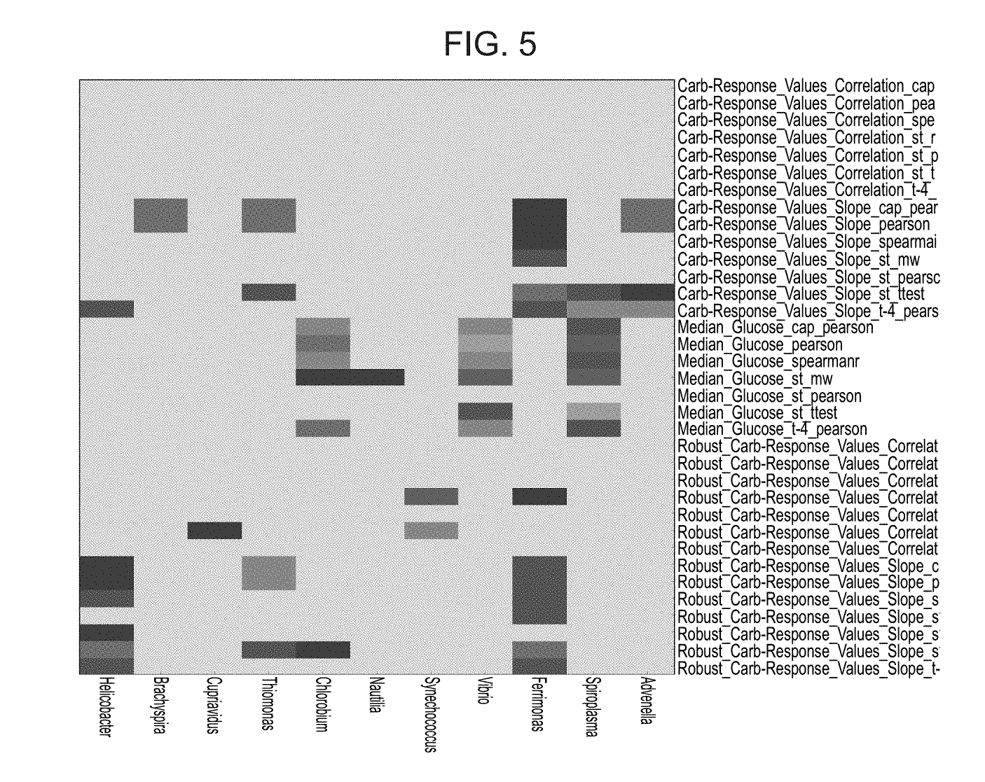

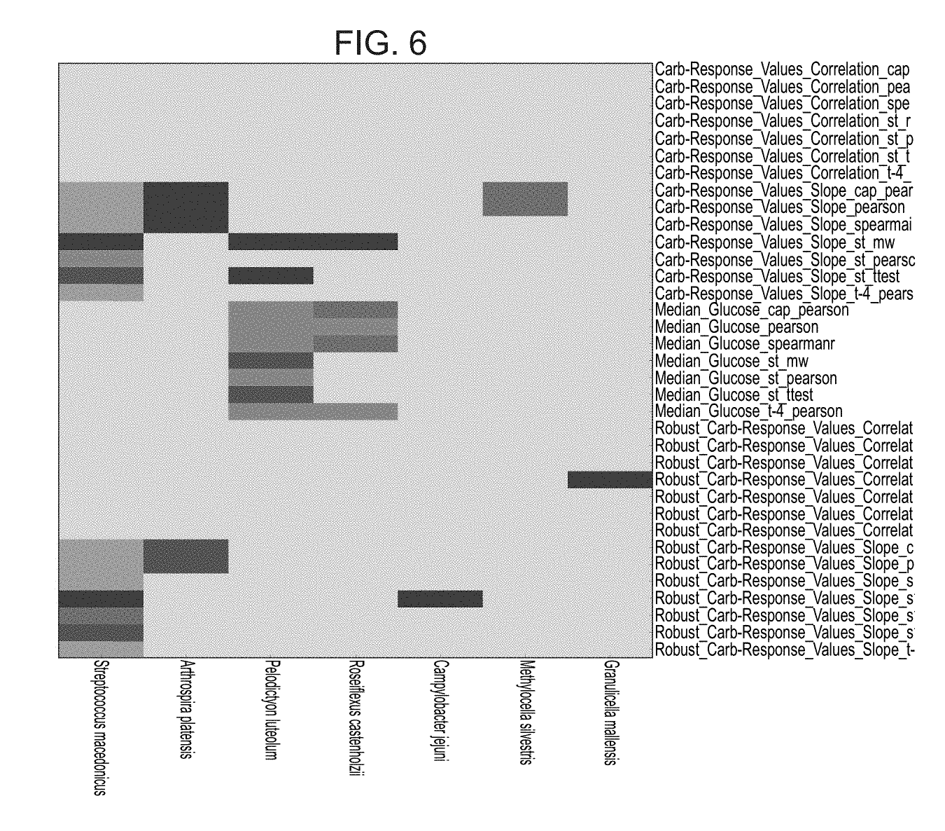

Each test was performed with different types of statistical tests (t-test, Mann-Whitney, Pearson and Spearman correlations) and corrected for multiple hypothesis testing using FDR. FIGS. 4-6 show the sets of bacteria significantly associated with the different features. Red indicates positive significant associations with the features, blue indicates negative significant associations. The associations were performed at the level of phylum, genus, species, and also at the level of KEGG metabolic pathways and modules.

### Example 3

### Measurements of Postprandial Responses, Clinical Data, and Gut Microbiome

### Materials and Methods

Study Design:

Study participants were healthy individuals aged 18-70 able to provide informed consent and operate a glucometer. Prior to the study, participants filled medical, lifestyle, and nutritional questionnaires. At connection week start, anthropometric, blood pressure and heart-rate measurements were taken by a CRA or a certified nurse, as well as a blood test. Glucose was measured for 7 days using the iPro2™ CGM with Enlite™ sensors (Medtronic, Minn., USA), independently calibrated with the Contour™ BGM (Bayer A G, Leverkusen, Germany) as required. During that week participants were instructed to record all daily activities, including meals and standardized meals, in real-time using their smartphones; meals were recorded with exact components and weights.

Standardized Meals.

Participants were given standardized meals (glucose, bread, bread and butter, bread and chocolate and fructose), calculated to have 50 g of available carbohydrates. Participants were instructed to consume these meals immediately after their night fast, not to modify the meal and to refrain from eating or performing strenuous physical activity before, and for two hours following consumption.

Stool Sample Collection.

Participants sampled their stool using detailed printed instructions. Sampling was done using a swab (N=776) or both a swab and an OMNIgene-GUT (OMR-200; DNA Genotek) stool collection kit (N=413, relative abundances (RA) for the same person are highly correlated (R=0.99 P<10⁻¹⁰) between swabs and OMNIgene-GUT collection methods). Collected samples were immediately stored in a home freezer (−20° C.), and transferred in a provided cooler to the investigators facilities where it was stored at −80° C. (−20° C. for OMNIIgene-GUT kits) until DNA extraction. All samples were taken within 3 days of connection week start.

Genomic DNA Extraction and Filtering.

Genomic DNA was purified using PowerMag Soil DNA isolation kit (MoBio) optimized for Tecan automated platform. For shotgun sequencing, 100 ng of purified DNA was sheared with a Covaris E220X sonicator. Illumina compatible libraries were prepared as described (Suez et al., 2014). For 16S rRNA sequencing, PCR amplification of the V3/4 region using the 515F/806R 16S rRNA gene primers was performed followed by 500 bp paired-end sequencing (Illumina MiSeq).

Microbial Analysis.

We used USearch8.0 (Edgar, 2013) to obtain RA from 16S rRNA reads. We filtered metagenomic reads containing Illumina adapters, filtered low quality reads and trimmed low quality read edges. We detected host DNA by mapping with GEM (Marco-Sola et al., 2012) to the Human genome with inclusive parameters, and removed those reads. We obtained RA from metagenomic sequencing via MetaPhlAn2 (Truong et al., 2015) with default parameters. We assigned length-normalized RA of genes, obtained by similar mapping with GEM to the reference catalog of (Li et al., 2014), to KEGG Orthology (KO) entries (Kanehisa and Goto, 2000), and these were then normalized to a sum of 1. We calculated RA of KEGG modules and pathways by summation. We considered only samples with >10K reads of 16S rRNA, and >10M metagenomic reads (>1.5M for daily samples in diet intervention cohort).

Associating PPGRs with Risk Factors and Microbiome Profile.

We calculated the median PPGR to standardized meals for each participant who consumed at least four of the standardized meals and correlated it with clinical parameters (Pearson). We also calculated the mean PPGR of replicates of each standardized meal (if performed) and correlated (Pearson) these values with (a) blood tests; (b) anthropometric measurements; (c) 16S rRNA RA at the species to phylum levels; (d) MetaPhlAn tag-level RA; and (e) RA of KEGG genes. We capped RA at a minimum of 1e-4 (16S rRNA), 1e-5 (MetaPhlAn) and 2e-7 (KEGG gene). For 16S rRNA analysis we removed taxa present in less than 20% of participants. Correlations on RAs was performed in logspace.

Enrichment analysis of higher phylogenetic levels (d) and KEGG pathways and modules (e) was performed by Mann-Whitney U-test between −log(P-value)*sign(R) of above correlations (d,e) of tags or genes contained in the higher order groups and −log(P-value)*sign(R) of the correlations of the rest of the tags or genes.

FDR Correction.

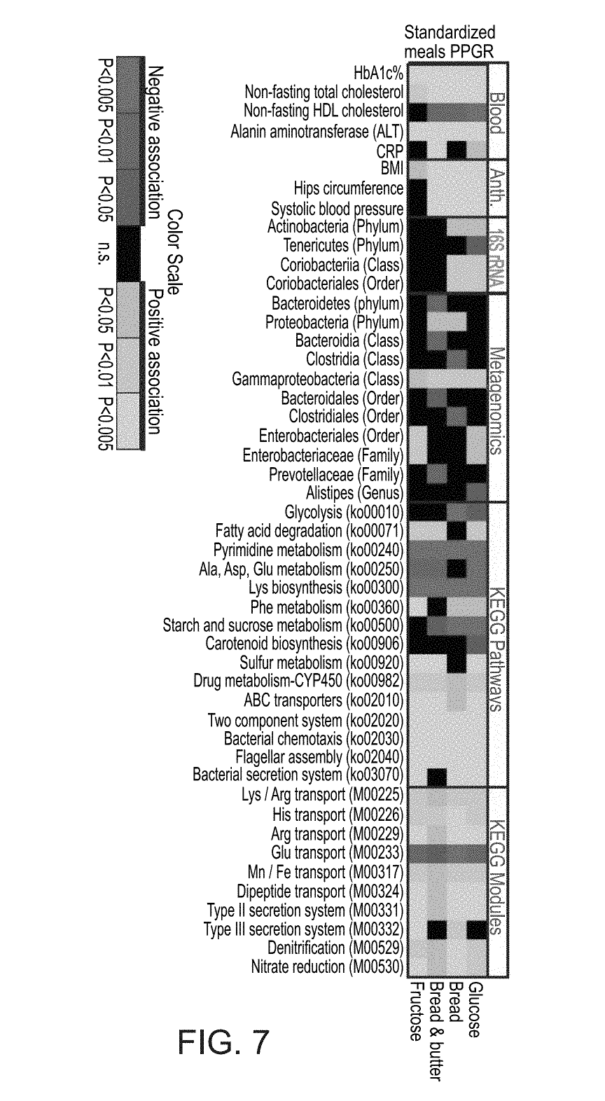

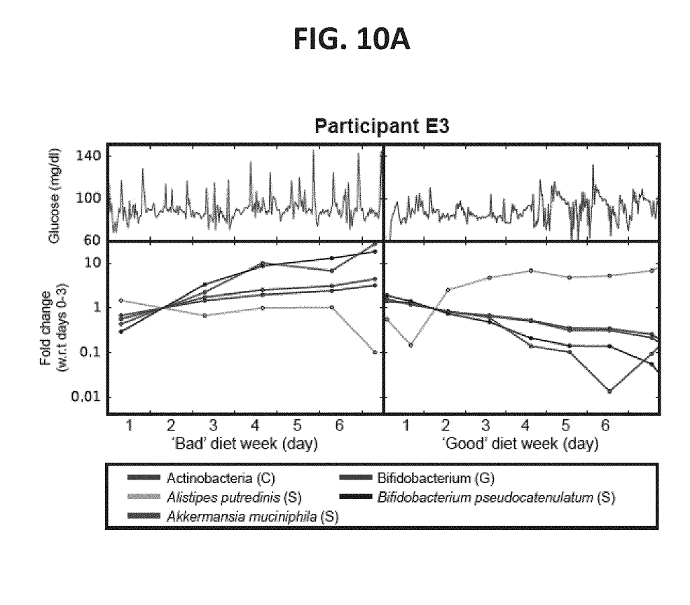

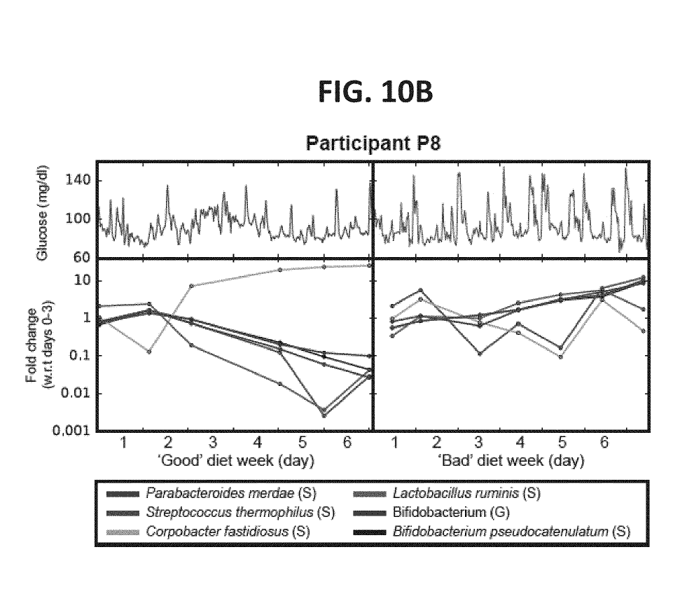

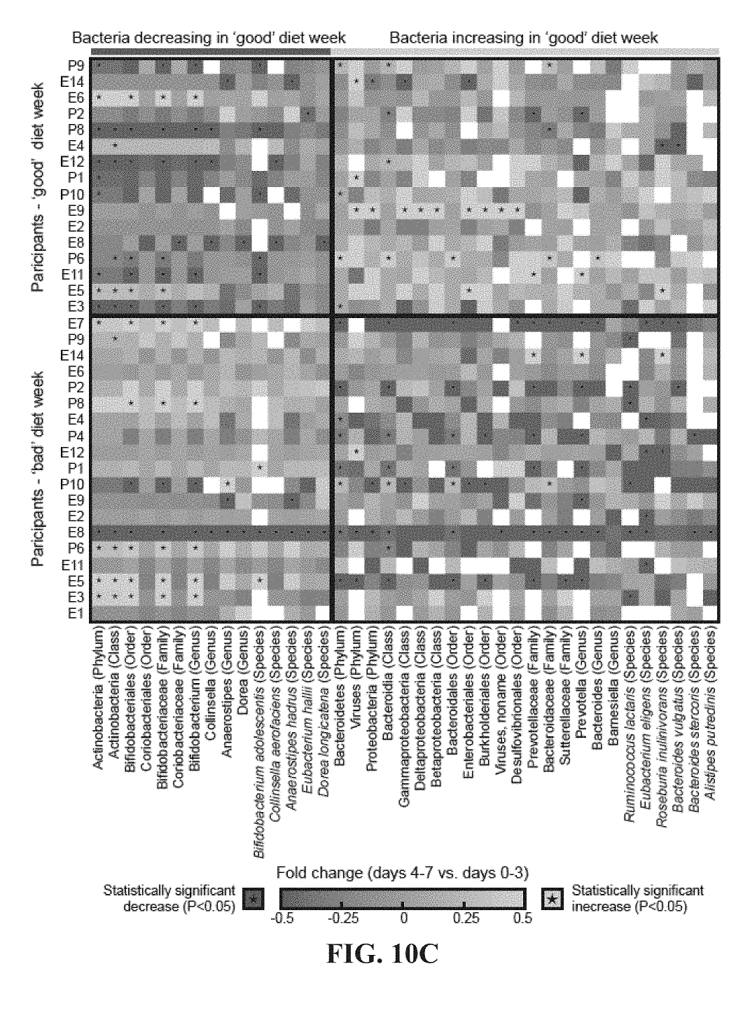

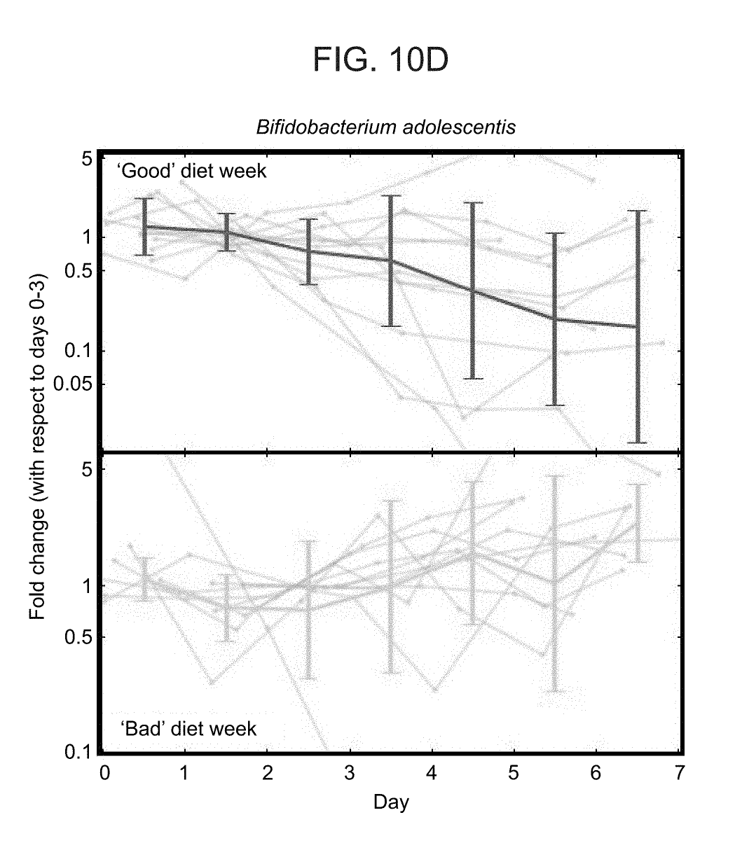

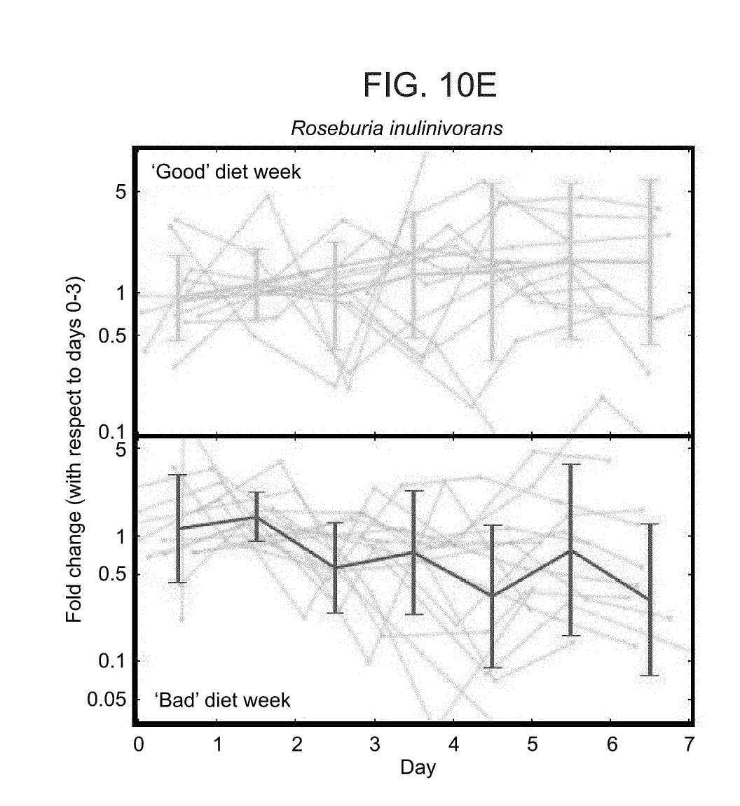

FDR was employed at the rate of 0.15, per tested variable (e.g., glucose standardized PPGR) per association test (e.g., with blood tests) for analyses in FIG. 7; per phylogenetic level in FIGS. 10A-E.

Meal Preprocessing.

We merged meals logged less than 30 minutes apart and removed meals logged within 90 minutes of other meals. We also removed very large (>1 kg) and very small (<15 g and <70 Calories) meals, meals with incomplete logging and meals consumed at the first and last 12 hours of the connection week.

PPGR Predictor.

Microbiome derived features were selected according to number of estimators using them in an additional predictor run on training data. We predicted PPGRs using stochastic gradient boosting regression, such that 80% of the samples and 40% of the features were randomly sampled for each estimator. The depth of the tree at each estimator was not limited, but leaves were restricted to have at least 60 instances (meals). We used 4000 estimators with a learning rate of 0.002.

Microbiome Changes During Dietary Intervention.

We determined the significantly changing taxa of each participant by a Z-test of fold-change in RA between the beginning and end of each intervention week against a null hypothesis of no change and standard deviation calculated from at least 25 fold changes across the first profiling week (no intervention) of corresponding taxa from all participants with similar initial RA. We checked whether a change was consistent across the cohort for each taxa by performing Mann-Whitney U-test between the Z statistics of the ‘good’ intervention weeks and those of the ‘bad’ intervention weeks across all participants.

### Results

To comprehensively characterize postprandial (post-meal) glycemic responses (PPGRs), 800 individuals were recruited aged 18-70 not previously diagnosed with TIIDM. The cohort is representative of the adult non-diabetic Israeli population (Israeli Center for Disease Control, 2014), with 54% overweight (BMI≥25 kg/m²), 22% obese (BMI≥30 kg/m²). These properties are also characteristic of the Western adult non-diabetic populations (World Health Organization, 2008).

Each participant was connected to a Continuous Glucose Monitor (CGM), which measures interstitial fluid glucose every 5 minutes for 7 full days (the “connection week”), using subcutaneous sensors. While connected to the CGM, participants were instructed to log their activities in real-time, including food intake, exercise and sleep. Each food item within every meal was logged along with its weight by selecting it from a database of 6,401 foods with full nutritional values based on the Israeli Ministry of Health database that we further improved and expanded with additional items from certified sources. During the connection week, participants were asked to follow their normal daily routine and dietary habits, except for the first meal of every day, which was provided as one of four different types of standardized meals, each consisting of 50 g of available carbohydrates. The PPGR of each meal was calculated by combining reported meal time with CGM data and computing the incremental area under the glucose curve in the two hours after the meal.

Prior to CGM connection, a comprehensive profile was collected from each participant, including: food-frequency, lifestyle and medical background questionnaires; anthropometrical measures (e.g., height, hip circumference); a panel of blood tests; and a single stool sample, used for microbiota profiling by both 16S rRNA and metagenomic sequencing.

Postprandial Glycemic Responses Associate with Multiple Risk Factors

The present data replicates known associations of PPGRs with risk factors, as the median standardized meal PPGR was significantly correlated with several known risk factors including BMI (R=0.24, P<10⁻¹⁰), glycated hemoglobin (HbA1c %, R=0.49, P<10⁻¹⁰), wakeup glucose (R=0.47, P<10⁻¹⁰), and age (R=0.42, P<10⁻¹⁰). These associations are not confined to extreme values but persist along the entire range of PPGR values, suggesting that the reduction in levels of risk factors is continuous across all postprandial values, with lower values being associated with lower levels of risk factors even within the normal value ranges.

High Interpersonal Variability in the Postprandial Response to Identical Meals

Next, the present inventors examined intra- and interpersonal variability in the PPGR to the same food. First, they assessed the extent to which PPGRs to three types of standardized meals which were given twice to every participant, are reproducible within the same person. Indeed, the two replicates showed high agreement (R=0.77 for glucose, R=0.77 for bread with butter, R=0.71 for bread, P<10⁻¹° in all cases), demonstrating that the PPGR to identical meals is reproducible within the same person, and that the present experimental system reliably measures this reproducibility. However, when comparing the PPGRs of different people to the same meal, high interpersonal variability was found, with the PPGRs of every meal type (except fructose) spanning the entire range of PPGRs measured in the cohort.

Next, the present inventors examined variability in the PPGRs to the multiple real-life meals reported by the participants. Since real-life meals vary in their amounts and may each contain several different food components, only meals that contained 20-40 g of carbohydrates and had a single dominant food component whose carbohydrate content exceeded 50% of the meal's carbohydrate content were examined. The resulting dominant foods that had at least 20 meal instances by their population-average glycemic PPGR were ranked. For foods with a published glycemic index, the instant population-average PPGRs agreed with published values (R=0.69, P<0.0005), further supporting the data.

Postprandial Variability is Associated with Clinical and Microbiome Profiles

Multiple significant associations between the standardized meal PPGRs of participants and both their clinical and gut microbiome data (FIG. 7 and Table 3). Notably, the TIIDM and metabolic syndrome risk factors HbA1c %, BMI, systolic blood pressure, and alanine aminotransferase (ALT) activity are all positively associated with PPGRs to all types of standardized meals, reinforcing the medical relevance of PPGRs. In most standardized meals, PPGRs also exhibit a positive correlation with CRP, whose levels rise in response to inflammation (FIG. 7).

TABLE 3

| | Positively correlated with | Negatively correlated with |
|---|---|---|
| | glycemic response - non | glycemic response - |
| | beneficial | beneficial |
| 16S | Coriobacteriia (16S C) | Tenericutes (16S P) |
| | Coriobacteriaceae (16S F) | |
| | Coriobacteriales (16S O) | |
| | Actinobacteria (16S P) | |
| Metagenomics | Gammaproteobacteria | Bacteroidia (MPA C) |
| (MPA) | (MPA C) | |
| | Enterobacteriaceae (MPA | Clostridia (MPA C) |
| | F) | |
| | Enterobacteriales (MPA | Prevotellaceae (MPA F) |
| | O) | |
| | Proteobacteria (MPA P) | Rikenellaceae (MPA F) |
| | | Alistipes (MPA G) |
| | | Bacteroidales (MPA O) |
| | | Clostridiales (MPA O) |
| | | Bacteroidetes (MPA P) |
| KEGG | M00032 | M00001 |
| modules | M00080 | M00002 |
| | M00095 | M00003 |
| | M00116 | M00004 |
| | M00136 | M00007 |
| | M00159 | M00014 |
| | M00191 | M00015 |
| | M00192 | M00016 |
| | M00208 | M00022 |
| | M00210 | M00026 |
| | M00212 | M00035 |
| | M00213 | M00048 |
| | M00215 | M00049 |
| | M00217 | M00051 |
| | M00219 | M00053 |
| | M00223 | M00055 |
| | M00225 | M00061 |
| | M00226 | M00082 |
| | M00229 | M00093 |
| | M00230 | M00096 |
| | M00232 | M00114 |
| | M00234 | M00129 |
| | M00241 | M00140 |
| | M00243 | M00144 |
| | M00249 | M00145 |
| | M00259 | M00149 |
| | M00273 | M00157 |
| | M00277 | M00177 |
| | M00278 | M00178 |
| | M00287 | M00179 |
| | M00300 | M00183 |
| | M00302 | M00184 |
| | M00303 | M00196 |
| | M00306 | M00205 |
| | M00317 | M00216 |
| | M00324 | M00233 |
| | M00331 | M00237 |
| | M00332 | M00239 |
| | M00333 | M00242 |
| | M00334 | M00244 |
| | M00336 | M00299 |
| | M00349 | M00319 |
| | M00356 | M00335 |
| | M00417 | M00338 |
| | M00447 | M00342 |
| | M00474 | M00345 |
| | M00506 | M00355 |
| | M00529 | M00357 |
| | M00530 | M00358 |
| | M00542 | M00359 |
| | M00545 | M00360 |
| | M00550 | M00373 |
| | M00551 | M00377 |
| | M00660 | M00390 |
| | | M00391 |
| | | M00422 |
| | | M00432 |
| | | M00525 |
| | | M00527 |
| | | M00549 |
| | | M00609 |
| | | M00631 |
| Kegg Pathways | ko00051 | ko00010 |
| | ko00052 | ko00030 |
| | ko00053 | ko00040 |
| | ko00071 | ko00061 |
| | ko00281 | ko00190 |
| | ko00310 | ko00196 |
| | ko00360 | ko00230 |
| | ko00362 | ko00240 |
| | ko00364 | ko00250 |
| | ko00380 | ko00253 |
| | ko00410 | ko00260 |
| | ko00440 | ko00270 |
| | ko00480 | ko00290 |
| | ko00591 | ko00300 |
| | ko00592 | ko00332 |
| | ko00625 | ko00400 |
| | ko00903 | ko00460 |
| | ko00910 | ko00471 |
| | ko00920 | ko00500 |
| | ko00982 | ko00510 |
| | ko01053 | ko00513 |
| | ko01220 | ko00520 |
| | ko02010 | ko00521 |
| | ko02020 | ko00524 |
| | ko02030 | ko00550 |
| | ko02040 | ko00563 |
| | ko02060 | ko00670 |
| | ko03070 | ko00680 |
| | ko04122 | ko00710 |
| | | ko00720 |
| | | ko00730 |
| | | ko00760 |
| | | ko00900 |
| | | ko00906 |
| | | ko00970 |
| | | ko00983 |
| | | ko01200 |
| | | ko01210 |
| | | ko01230 |
| | | ko03008 |
| | | ko03010 |
| | | ko03015 |
| | | ko03018 |
| | | ko03020 |
| | | ko03022 |
| | | ko03030 |
| | | ko03040 |
| | | ko03050 |
| | | ko03060 |
| | | ko03410 |
| | | ko03420 |
| | | ko03430 |
| | | ko03440 |
| | | ko04010 |
| | | ko04110 |
| | | ko04111 |
| | | ko04112 |
| | | ko04113 |
| | | ko04114 |
| | | ko04120 |
| | | ko04141 |
| | | ko04142 |
| | | ko04144 |
| | | ko04145 |
| | | ko04150 |
| | | ko04151 |
| | | ko04152 |
| | | ko04390 |
| | | ko04391 |
| | | ko04530 |

With respect to microbiome features, the phylogenetically related Proteobacteria and Enterobacteriaceae both exhibit positive associations with a few of the standardized meals PPGR (FIG. 7). These taxa have reported associations with poor glycemic control, and with components of the metabolic syndrome including obesity, insulin resistance and impaired lipid profile (Xiao et al., 2014). RAs of Actinobacteria are positively associated with the PPGR to both glucose and bread, which is intriguing since high levels of this phylum were reported to associate with a high-fat low-fiber diet (Wu et al., 2011).

At the functional level, the KEGG pathways of bacterial chemotaxis and of flagellar assembly, reported to increase in mice fed high-fat diets and decrease upon prebiotics administration (Everard et al., 2014), exhibit positive associations with several standardized meal PPGRs (FIG. 7). The KEGG pathway of ABC transporters, reported to be positively associated with TIIDM (Karlsson et al., 2013) and with a western high-fat/high-sugar diet (Turnbaugh et al., 2009), also exhibits positive association with several standardized meal PPGRs (FIG. 7). Several bacterial secretion systems, including both type 2 and type 3 secretion systems that are instrumental in bacterial infection and quorum sensing (Sandkvist, 2001) are positively associated with most standardized meal PPGRs (FIG. 7). Finally, KEGG modules for transport of the positively charged amino acids lysine and arginine are associated with high PPGR to standardized foods, while transport of the negatively charged amino acid glutamate is associated with low PPGRs to these foods.

Taken together, these results show that PPGRs vary greatly across different people and associates with multiple person-specific clinical and microbiome factors.

Prediction of Personalized Postprandial Glycemic Responses

The present inventors next asked whether clinical and microbiome factors could be integrated into an algorithm that predicts individualized PPGRs. To this end, a two-phase approach was employed. In the first, discovery phase, the algorithm was developed on the main cohort of 800 participants, and performance was evaluated using a standard leave-one-out cross validation scheme, whereby PPGRs of each participant were predicted using a model trained on the data of all other participants. In the second, validation phase, an independent cohort of 100 participants was recruited and profiled, and their PPGRs were predicted using the model trained only on the main cohort.

Given non-linear relationships between PPGRs and the different factors, we devised a model based on gradient boosting regression (Friedman, 2001). This model predicts PPGRs using the sum of thousands of different decision trees. Trees are inferred sequentially, with each tree trained on the residual of all previous trees and making a small contribution to the overall prediction. The features within each tree are selected by an inference procedure from a pool of 137 features representing meal content (e.g., energy, macronutrients, micronutrients); daily activity (e.g., meal, exercise, sleep times); blood parameters (e.g., HbA1c %, HDL cholesterol); CGM-derived features; questionnaires; and microbiome features (16S rRNA and metagenomic RAs, KEGG pathway and module RAs and bacterial growth dynamics—PTRs Korem et al., 2015).

As a baseline reference, the ‘carbohydrate counting’ model was used, as it is the current gold standard for predicting PPGRs (American Diabetes Association., 2015b; Bao et al., 2011). On the present data, this model that consists of a single explanatory variable representing the meal's carbohydrate amount achieves a modest yet statistically significant correlation with PPGRs (R=0.38, P<10⁻¹⁰). A model using only meal caloric content performs worse (R=0.33, P<10⁻¹⁰). The presently developed predictor that integrates the above person-specific factors predicts the held-out PPGRs of individuals with a significantly higher correlation (R=0.68, P<10⁻¹⁰). This correlation approaches the presumed upper bound limit set by the 0.71-0.77 correlation that was observed between the PPGR of the same person to two replicates of the same standardized meal.

### Validation of Personalized Postprandial Glycemic Response Predictions on an Independent Cohort

The model was validated on an independent cohort of 100 individuals that were recruited separately.

Notably, the algorithm, derived solely using the main 800 participants cohort, achieved similar performance on the 100 participants of the validation cohort (R=0.68 & R=0.70 on the main and validation cohorts, respectively). The reference carbohydrate counting model achieved the same performance as in the main cohort (R=0.38). This result further supports the ability of the algorithm to provide personalized PPGR predictions.

### Factors Underlying Personalized Postprandial Responses

To gain insight into the contribution of the different features in the algorithm's predictions, partial dependence plots (PDP) were examined. These are commonly used to study functional relations between features used in predictors such as the gradient boosting regressor and an outcome (PPGRs in our case; Hastie et al., 2008). PDPs graphically visualize the marginal effect of a given feature on prediction outcome after accounting for the average effect of all other features.

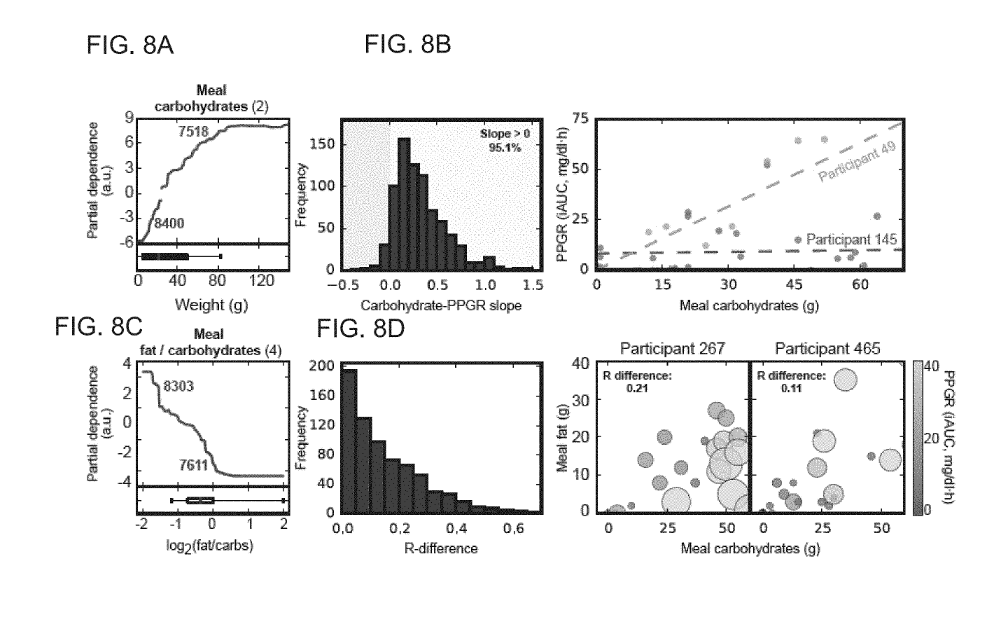

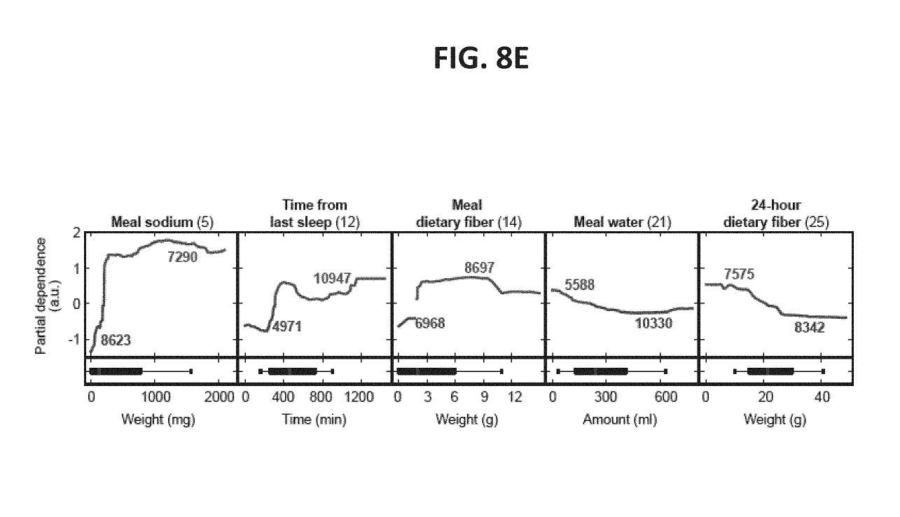

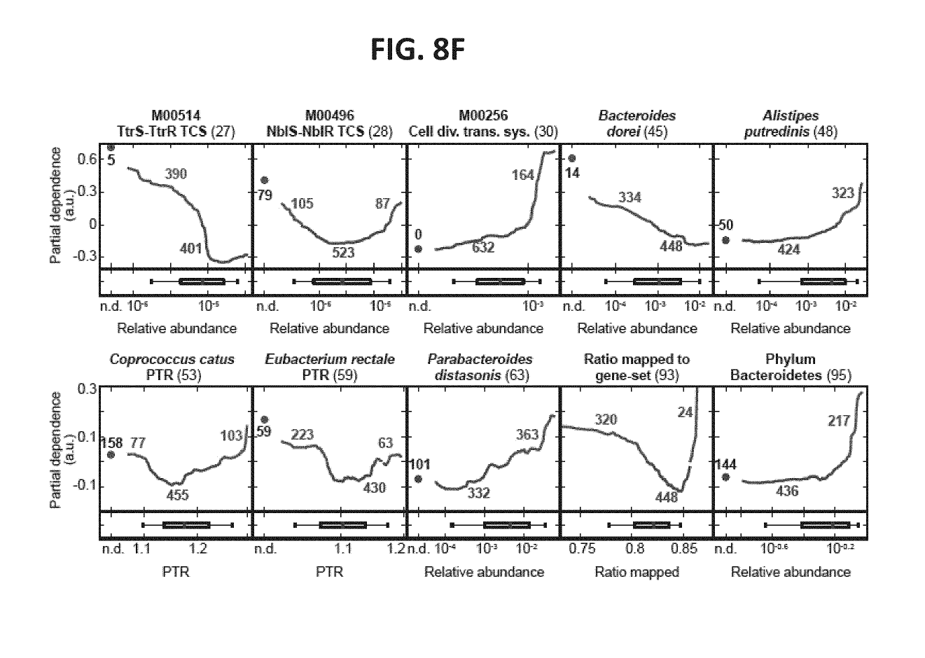

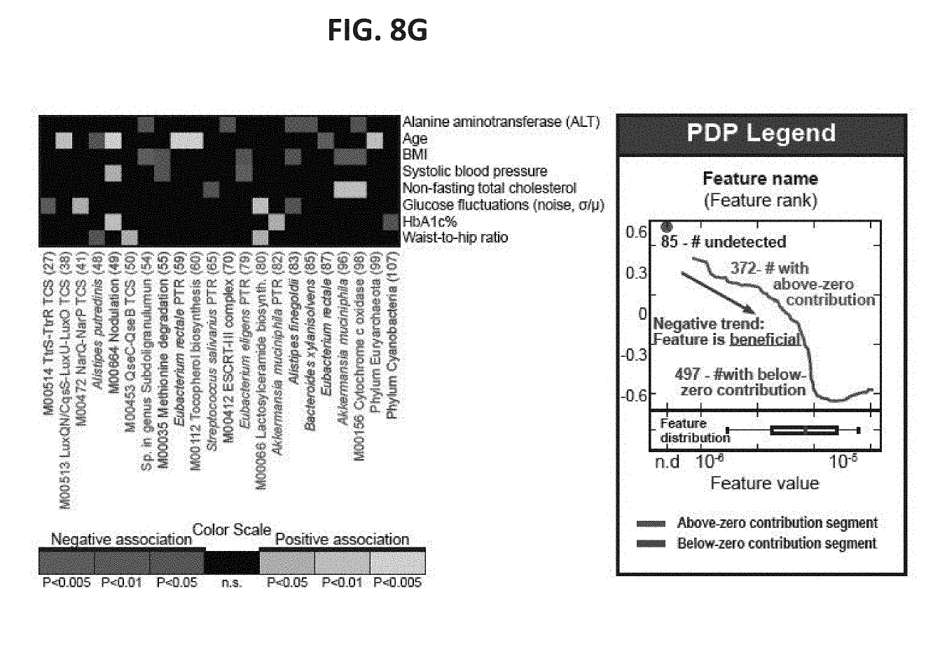

As expected, the PDP of carbohydrates (FIG. 8A) shows that as the meal carbohydrate content increases, the algorithm predicts, on average, a higher PPGR. This relation, of higher predicted PPGR with increasing feature value, may be termed non-beneficial (with respect to prediction), and the opposite relation, of lower predicted PPGR with increasing feature value, may be termed beneficial (also with respect to prediction; see PDP legend in FIGS. 8A-G). However, since PDPs display the overall contribution of each feature across the entire cohort, the present inventors asked whether the relationship between carbohydrate amount and PPGRs varies across people. To this end, for each participant the slope of the linear regression between the PPGR and carbohydrate amount of all his/her meals was computed. As expected, this slope was positive for nearly all (95.1%) participants, reflective of higher PPGRs in meals richer in carbohydrates. However, the magnitude of this slope varies greatly across the cohort, with the PPGR of some people correlating well with the carbohydrate content (i.e., carbohydrates “sensitive”) and that of others exhibiting equally high PPGRs but little relationship to the amount of carbohydrates (carbohydrate “insensitive”; FIG. 8B). This result suggests that carbohydrate sensitivity is also person-specific.

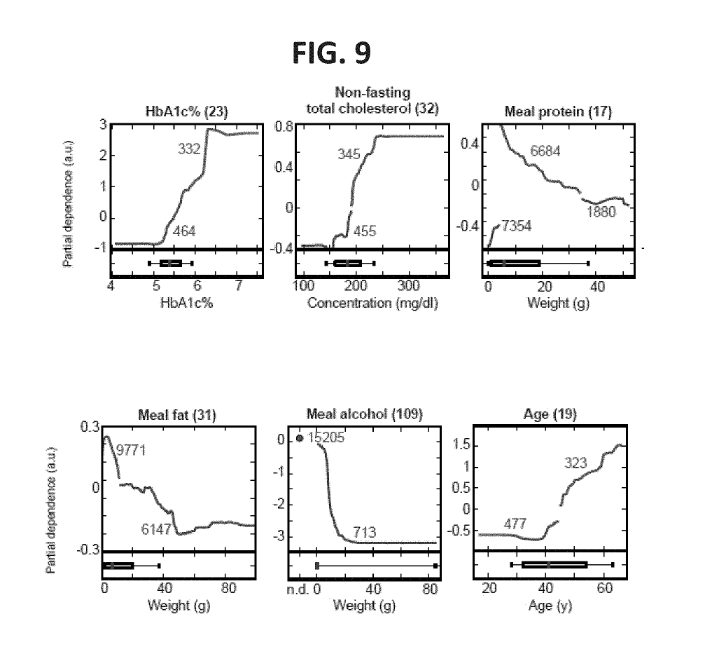

The PDP of fat shows a beneficial effect for fat since the present algorithm predicts, on average, lower PPGR as the meal's ratio of fat to carbohydrates (FIG. 8C) or total fat content (FIG. 9) increases, consistent with studies showing that adding fat to meals may reduce the PPGR (Cunningham and Read, 1989). However, here too, it was found that the effect of fat varies across people. The present inventors compared the explanatory power of a linear regression between each participant's PPGR and meal carbohydrates, with that of regression using both fat and carbohydrates. They then used the difference in Pearson R between the two models as a quantitative measure of the added contribution of fat (FIG. 8D). For some participants a reduction in PPGR was observed with the addition of fat, while for others meal fat content did not add much to the explanatory power of the regressor based only on the meal's carbohydrates content (FIG. 8D).

Interestingly, while dietary fibers in the meal increase the predicted PPGR, their long-term effect is beneficial as higher amount of fibers consumed in the 24 hours prior to the meal reduces the predicted PPGR (FIG. 8E). The meal's sodium content, the time that passed since last sleeping, and a person's cholesterol levels or age all exhibit non-beneficial PDPs, while the PDPs of the meal's alcohol content and the amount of water contained in the meal all display beneficial effects (FIGS. 8E, 9). As expected, the PDP of HbA1c % shows a non-beneficial effect with increased PPGR at higher HbA1c % values; intriguingly, higher PPGRs are predicted, on average, for individuals with HbA1c % above ˜5.5%, which is very close to the prediabetes threshold of 5.7%.

A full list of beneficial and non-beneficial bacteria derived from the output of the personalized response predictor is presented in Table 4 herein below.

TABLE 4

| Non-Beneficial | Beneficial |
|---|---|
| 16S_phylum:Actinobacteria’ | ‘16S_phylum:Cyanobacteria’ |
| ‘16S_phylum:Bacteroidetes’ | ‘16S_phylum:Lentisphaerae’ |
| ‘16S_phylum:Euryarchaeota’ | ‘16S_phylum:Proteobacteria’ |
| ‘16S_phylum:Fusobacteria’ | ‘16S_phylum:Verrucomicrobia’ |
| ‘PTR of Akkermansia muciniphila’ | ‘PTR of Eubacterium rectale’ |
| ‘PTR of Eubacterium eligens’ | ‘KEGG Module - M00035 Methionine |
| | degradation’ |
| ‘PTR of Ruminococcus bromii’ | ‘KEGG Module - M00040 Tyrosine |
| | biosynthesis, prephanate => pretyrosine => |
| | tyrosine’ |
| ‘PTR of Streptococcus salivarius’ | ‘KEGG Module - M00053 Pyrimidine |
| | deoxyribonucleotide biosynthesis, |
| | CDP/CTP => dCDP/dCTP, dTDP/dTTP’ |
| ‘KEGG Module - M00066 | ‘KEGG Module - M00343 Archaeal |
| Lactosylceramide biosynthesis’ | proteasome’ |
| ‘KEGG Module - M00092 | ‘KEGG Module - M00411 SCF-GRR1 |
| Phosphatidylethanolamine (PE) | complex’ |
| biosynthesis, ethanolamine => PE’ | |
| ‘KEGG Module - M00112 | ‘KEGG Module - M00412 ESCRT-III |
| Tocopherol/tocotorienol biosynthesis’ | complex’ |
| ‘KEGG Module - M00156 Cytochrome c | ‘KEGG Module - M00496 ComD-ComE |
| oxidase, cbb3-type’ | (competence) two-component regulatory |
| | system’ |
| ‘KEGG Module - M00256 Cell division | ‘KEGG Module - M00497 GlnL-GlnG |
| transport system’ | (nitrogen regulation) two-component |
| | regulatory system’ |
| ‘KEGG Module - M00453 QseC-QseB | ‘KEGG Module - M00514 TtrS-TtrR |
| (quorum sensing) two-component | (tetrathionate respiration) two-component |
| regulatory system’ | regulatory system’ |
| ‘KEGG Module - M00468 SaeS-SaeR | ‘KEGG Module - M00664 Nodulation’ |
| (staphylococcal virulence regulation) | |
| two-component regulatory system’ | |
| ‘KEGG Module - M00470 YxdK-YxdJ | ‘MetaPhlAn - s_Alistipes_finegoldii’ |
| (antimicrobial peptide response) two- | |
| component regulatory system’ | |
| ‘KEGG Module - M00472 NarQ-NarP | ‘MetaPhlAn - s_Alistipes_senegalensis’ |
| (nitrate respiration) two-component | |
| regulatory system’ | |
| ‘KEGG Module - M00505 KinB-AlgB | ‘MetaPhlAn - s_Bacteroides_dorei’ |
| (alginate production) two-component | |
| regulatory system’ | |
| ‘KEGG Module - M00513 LuxQN/CqsS- | ‘MetaPhlAn - |
| LuxU-LuxO (quorum sensing) two- | s_Bacteroides_xylanisolvens’: Beneficial, |
| component regulatory system’ | |
| ‘MetaPhlAn - | ‘MetaPhlAn - s_Eubacterium_rectale’: |
| s_Akkermansia_muciniphila’ | |
| ‘MetaPhlAn - s_Alistipes_putredinis’ | ‘MetaPhlAn - s_Roseburia_inulinivorans’ |
| ‘MetaPhlAn - | ‘16S_phylum:Cyanobacteria’ |
| s_Bacteroides_thetaiotaomicron’ | |
| ‘MetaPhlAn - s_Eubacterium_siraeum’ | |
| ‘MetaPhlAn - | |
| s_Parabacteroides_distasonis’ | |
| ‘MetaPhlAn - s_Ruminococcus_bromii’ | |
| ‘MetaPhlAn - | |
| s_Subdoligranulum_unclassified’ | |
| 16S_phylum:Actinobacteria’ | |
| ‘16S_phylum:Bacteroidetes’ | |
| ‘16S_phylum:Euryarchaeota’ | |
| ‘KEGG Module - M00065 GPI-anchor | |
| biosynthesis, core oligosaccharide’ | |
| ‘KEGG Module - M00389 APC/C | |
| complex’ | |

The 72 PDPs of the microbiome-based features used in the predictor were either beneficial (21 factors), non-beneficial (28), or non-decisive (23) in that they mostly decreased, increased, or neither, as a function of the microbiome feature. The resulting PDPs had several intriguing trends. For example, growth of Eubacterium rectale was mostly beneficial, as in 430 participants with high inferred growth for E. rectale it associates with a lower PPGR (FIG. 8F and Table 4 herein above). RAs of Parabacteroides distasonis were found non-beneficial by the predictor (FIG. 8F and Table 4 herein above). As another example, the KEGG module of cell-division transport system (M00256) was non-beneficial, and in the 164 participants with the highest levels for it, it associates with a higher PPGR (FIG. 8F and Table 4 herein above). Bacteroides thetaiotaomicron was non-beneficial (Table 4 herein above), and it was associated with obesity. In the case of Alistipes putredinis and the Bacteroidetes phylum, the non-beneficial classification that the predictor assigns to both of them is inconsistent with previous studies that found them to be negatively associated with obesity (Ridaura et al., 2013; Turnbaugh et al., 2006).

To assess the clinical relevance of the microbiome-based PDPs, the present inventors computed the correlation between several risk factors and overall glucose parameters, and the factors with beneficial and non-beneficial PDPs across the entire 800-person cohort. 20 statistically significant correlations (P<0.05, FDR corrected) where microbiome factors termed non-beneficial correlated with risk factors, and those termed beneficial exhibited an anti-correlation (FIG. 8G and Table 4 herein above). For example, higher levels of the beneficial methionine degradation KEGG module (M00035) resulted in lower PPGRs in our algorithm, and across the cohort, this bacteria anti-correlates with systolic blood pressure and with BMI (FIG. 8G and Table 4 herein above). Similarly, fluctuations in glucose levels across the connection week correlates with nitrate respiration two-component regulatory system (M00472) and with lactosylceramide biosynthesis (M00066), which were both termed non-beneficial. Glucose fluctuations also anti-correlate with level of the tetrathionate respiration two-component regulatory system (M00514) and with RAs of Alistipes finegoldii, both termed beneficial (FIG. 8G and Table 4 herein above). In 14 other cases, factors with beneficial or non-beneficial PDPs were correlated and anti-correlated with risk factors, respectively.

These results suggest that PPGRs are associated with multiple and diverse factors, including factors unrelated to meal content.

Personally Tailored Dietary Interventions Improve Postprandial Responses

Next, the present inventors asked whether personally tailored dietary interventions based on the algorithm could improve PPGRs. A two-arm blinded randomized controlled trial was designed and 26 new participants were recruited. A clinical dietitian met each participant and compiled 4-6 distinct isocaloric options for each type of meal (breakfast, lunch, dinner, and up to two intermediate meals), accommodating the participant's regular diet, eating preferences, and dietary constraints. Participants then underwent the same one-week profiling of the main 800-person cohort (except that they consumed the meals compiled by the dietitian), thus providing the inputs (microbiome, blood parameters, CGM, etc.) that the algorithm needs for predicting their PPGRs.

Participants were then blindly assigned to one of two arms. In the first, “prediction arm”, the algorithm in a leave-one-out scheme was applied to rank every meal of each participant in the profiling week (i.e., the PPGR to each predicted meal was hidden from the predictor). These rankings were then used to design two one-week diets: (1) a diet composed of the meals predicted by the algorithm to have low PPGRs (the ‘good’ diet); and (2) a diet composed of the meals with high predicted PPGRs (the ‘bad’ diet). Every participant then followed each of the two diets for one full week, during which he/she was connected to a CGM and a daily stool sample was collected (if available). The order of the two diet weeks was randomized for each participant and the identity of the intervention weeks (i.e., whether they are ‘good’ or ‘bad’) was kept blinded from CRAs, dietitians and participants.

The second, “expert arm”, was used as a gold standard for comparison. Participants in this arm underwent the same process as the prediction arm except that instead of using the predictor for selecting their ‘good’ and ‘bad’ diets a clinical dietitian and a researcher experienced in analyzing CGM data (collectively termed “expert”) selected them based on their measured PPGRs to all meals during the profiling week. Specifically, meals that according to the expert's analysis of their CGM had low and high PPGRs in the profiling week were selected for the ‘good’ and ‘bad’ diets, respectively. Thus, to the extent that PPGRs are reproducible within the same person, this expert-based arm should result in the largest differences between the ‘good’ and ‘bad’ diets because the selection of meals in the intervention weeks is based on their CGM data.

Notably, for 10 of the 12 participants of the predictor-based arm, PPGRs in the ‘bad’ diet were significantly higher than in the ‘good’ diet (P<0.05). Differences between the two diets are also evident in fewer glucose spikes and fewer fluctuations in the raw weeklong CGM data. The success of the predictor was comparable to that of the expert-based arm, in which significantly lower PPGRs in the ‘good’ versus the ‘bad’ diet were observed for 8 of its 14 participants (P<0.05, 11 of 14 participants with P<0.1).

When combining the data across all participants, the ‘good’ diet had significantly lower PPGRs than the ‘bad’ diet (P<0.05) as well as improvement in other measures of blood glucose metabolism in both study arms, specifically, lower fluctuations in glucose levels across the CGM connection week (P<0.05), and a lower maximal PPGR (P<0.05) in the ‘good’ diet.

Both study arms constitute personalized nutritional interventions and thus demonstrate the efficacy of this approach in lowering PPGRs. However, the predictor-based approach has broader applicability since it can predict PPGRs to arbitrary unseen meals, whereas the ‘expert’-based approach will always require CGM measurements of the meals it prescribes.

Post-hoc examination of the prescribed diets revealed the personalized aspect of the diets in both arms in that multiple dominant food components prescribed in the ‘good’ diet of some participants were prescribed in the ‘bad’ diet of. This occurs when components induced opposite CGM-measured PPGRs across participants (expert arm) or were predicted to have opposite PPGRs (predictor arm).

The correlation between the measured PPGR of meals during the profiling week and the average CGM-measured PPGR of the same meals during the dietary intervention was 0.70, which is similar to the reproducibility observed for standardized meals (R=0.71-0.77). Thus, as in the case of standardized meals, a meal's PPGR during the profiling week was not identical to its PPGR in the dietary intervention week. Notably, using only the first profiling week data of each participant, our algorithm predicted the average PPGRs of meals in the dietary intervention weeks with an even higher correlation (R=0.80). Since the predictor also incorporates context-specific factors (e.g., previous meal content, time since sleep), this result also suggests that such factors may be important determinants of PPGRs.

Taken together, these results show the utility of personally-tailored dietary interventions for improving PPGRs in a short term intervention period, and the ability of the present algorithm to devise such interventions.

Alterations in Gut Microbiota Following Personally Tailored Dietary Interventions

Finally, the daily microbiome samples collected during the intervention weeks were used to ask whether the interventions induced significant changes in the gut microbiota. Previous studies showed that even short-term dietary interventions of several days may significantly alter the gut microbiota (David et al., 2014; Korem et al., 2015).

The present inventors detected changes following the dietary interventions that were significant relative to a null hypothesis of no change derived from the first week, in which there was no intervention, across all participants (FIGS. 10A,B). While many of these significant changes were person-specific, several taxa changed consistently in most participants (P<0.05, FDR corrected, FIG. 10C, Table 5 herein below). Moreover, in most cases in which the consistently changing taxa had reported associations in the literature, the direction of change in RA following the ‘good’ diet was consistent with reported beneficial associations. For example, low levels of Bifidobacterium adolescentis, reported to be associated with greater weight loss (Santacruz et al., 2009), generally decrease in RA following the ‘good’ diet and increase following the ‘bad’ diet (FIGS. 10C-D). Similarly, TIIDM has been associated with low levels of Roseburia inulinivorans (Qin et al., 2012) (FIG. 10E), Eubacterium eligens (Karlsson et al., 2013), and Bacteroides vulgatus (Ridaura et al., 2013), and all these bacteria increase following the ‘good’ diet and decrease following the ‘bad’ diet (FIG. 10C). The Bacteroidetes phylum, for which low levels associate with obesity and high fasting glucose (Turnbaugh et al., 2009), increases following the ‘good’ diet and decreases following the ‘bad’ diet (FIG. 10C). Low levels of Anaerostipes associate with improved glucose tolerance and reduced plasma triglyceride levels in mice (Everard et al., 2011) and indeed these bacteria decrease following the ‘good’ diet and increase following the ‘bad’ diet (FIG. 10C). Finally, low levels of Alistipes putredinis associate with obesity (Ridaura et al., 2013) and this bacteria increased following the ‘good’ diet (FIG. 10C).

These findings demonstrate that while both baseline microbiota composition and personalized dietary intervention vary between individuals, several consistent microbial changes may be induced by dietary intervention with consistent effect on PPGR.

TABLE 5

| Non-Beneficial | Beneficial |
|---|---|
| Actinobacteria (P) | Bacteroidetes (P) |
| Firmicutes (P) | Verrucomicrobia (P) |
| Actinobacteria (C) | Viruses noname (P) |
| Bacilli (C) | Proteobacteria (P) |
| Clostridia (C) | Bacteroidia (C) |
| Bifidobacteriales (O) | Verrucomicrobiae (C) |
| Lactobacillales (O) | Viruses noname (C) |
| Verrucomicrobiales (O) | Negativicutes (C) |
| Coriobacteriales (O) | Gammaproteobacteria (C) |
| Clostridiales (O) | Erysipelotrichia (C) |
| Bifidobacteriaceae (F) | Deltaproteobacteria (C) |
| Streptococcaceae (F) | Betaproteobacteria (C) |
| Lactobacillaceae (F) | Bacteroidales (O) |
| Verrucomicrobiaceae (F) | Selenomonadales (O) |
| Coriobacteriaceae (F) | Enterobacteriales (O) |
| Ruminococcaceae (F) | Burkholderiales (O) |
| Lachnospiraceae (F) | Erysipelotrichales (O) |
| Bifidobacterium (G) | Viruses noname (O) |
| Streptococcus (G) | Desulfovibrionales (O) |
| Ruminococcus (G) | Prevotellaceae (F) |
| Clostridium (G) | Clostridiaceae (F) |
| Lachnospiraceae noname (G) | Enterobacteriaceae (F) |
| Collinsella (G) | Bacteroidaceae (F) |
| Anaerostipes (G) | Peptostreptococcaceae (F) |
| Faecalibacterium (G) | Bacteroidales noname (F) |
| Subdoligranulum (G) | Eubacteriaceae (F) |
| Dorea (G) | Sutterellaceae (F) |
| Coprococcus (G) | Erysipelotrichaceae (F) |
| Oscillibacter (G) | Rikenellaceae (F) |
| Blautia (G) | Oscillospiraceae (F) |
| Streptococcus thermophilus (S) | Porphyromonadaceae (F) |
| Roseburia intestinalis (S) | Desulfovibrionaceae (F) |
| Bifidobacterium adolescentis (S) | Prevotella (G) |
| Lachnospiraceae bacterium | Peptostreptococcaceae noname (G) |
| 1 1 57FAA (S) | |
| Bacteroides cellulosilyticus (S) | Odoribacter (G) |
| Ruminococcus sp 5 1 39BFAA (S) | Escherichia (G) |
| Ruminococcus bromii (S) | Roseburia (G) |
| Peptostreptococcaceae noname | Bacteroides (G) |
| unclassified (S) | |
| Bifidobacterium longum (S) | Bacteroidales noname (G) |
| Eubacterium rectale (S) | Eubacterium (G) |
| Bacteroides caccae (S) | Adlercreutzia (G) |
| Roseburia hominis (S) | Erysipelotrichaceae noname (G) |
| Lachnospiraceae bacterium | Bilophila (G) |
| 5 1 63FAA (S) | |
| Eubacterium ventriosum (S) | Alistipes (G) |
| Faecalibacterium prausnitzii (S) | Parabacteroides (G) |
| Parabacteroides merdae (S) | Barnesiella (G) |
| Anaerostipes hadrus (S) | Prevotella copri (S) |
| Collinsella aerofaciens (S) | Escherichia coli (S) |
| Parabacteroides distasonis (S) | Lachnospiraceae bacterium |
| | 8 1 57FAA (S) |
| Eubacterium hallii (S) | Ruminococcus lactaris (S) |
| Dorea longicatena (S) | Eubacterium eligens (S) |
| Bilophila unclassified (S) | Roseburia inulinivorans (S) |
| Subdoligranulum unclassified (S) | Bacteroidales bacterium ph8 (S) |
| Coprococcus catus (S) | Bacteroides dorei (S) |
| Oscillibacter unclassified (S) | Bacteroides uniformis (S) |
| Ruminococcus obeum (S) | Bacteroides thetaiotaomicron (S) |
| Dorea formicigenerans (S) | Clostridium bartlettii (S) |
| Ruminococcus torques (S) | Bacteroides vulgatus (S) |
| Alistipes shahii (S) | Bacteroides massiliensis (S) |
| | Bacteroides stercoris (S) |
| | Barnesiella intestinihominis (S) |
| | Bacteroides ovatus (S) |
| | Coprococcus comes (S) |
| | Alistipes putredinis (S) |
| | Eubacterium ramulus (S) |

P, phylum;
C, class;
O, order;
F, family;
G, genus;
S, species.

### REFERENCES

American Diabetes Association. (2015a). 5. Prevention or Delay of Type 2 Diabetes. Diabetes Care 38, S31-S32.
American Diabetes Association. (2015b). 4. Foundations of Care: Education, Nutrition, Physical Activity, Smoking Cessation, Psychosocial Care, and Immunization. Diabetes Care 38, 520-530.
Atkinson, F. S., Foster-Powell, K., and Brand-Miller, J. C. (2008). International tables of glycemic index and glycemic load values: 2008. Diabetes Care 31, 2281-2283.
Bailey, T. S., Ahmann, A., Brazg, R., Christiansen, M., Garg, S., Watkins, E., Welsh, J. B., and Lee, S. W. (2014). Accuracy and acceptability of the 6-day Enlite continuous subcutaneous glucose sensor. Diabetes Technol. Ther. 16, 277-283.
Bansal, N. (2015). Prediabetes diagnosis and treatment: A review. World J. Diabetes 6, 296-303.
Bao, J., Gilbertson, H. R., Gray, R., Munns, D., Howard, G., Petocz, P., Colagiuri, S., and Brand-Miller, J. C. (2011). Improving the Estimation of Mealtime Insulin Dose in Adults With Type 1 Diabetes: The Normal Insulin Demand for Dose Adjustment (NIDDA) study. Diabetes Care 34, 2146-2151.
Blaak, E. E., Antoine, J.-M., Benton, D., Björck, I., Bozzetto, L., Brouns, F., Diamant, M., Dye, L., Hulshof, T., Holst, J. J., et al. (2012). Impact of postprandial glycaemia on health and prevention of disease. Obes. Rev. 13, 923-984.
Carpenter, D., Dhar, S., Mitchell, L. M., Fu, B., Tyson, J., Shwan, N. A. A., Yang, F., Thomas, M. G., and Armour, J. A. L. (2015). Obesity, starch digestion and amylase: association between copy number variants at human salivary (AMY1) and pancreatic (AMY2) amylase genes. Hum. Mol. Genet. 24, 3472-3480.
Cavalot, F., Pagliarino, A., Valle, M., Di Martino, L., Bonomo, K., Massucco, P., Anfossi, G., and Troyati, M. (2011). Postprandial Blood Glucose Predicts Cardiovascular Events and All-Cause Mortality in Type 2 Diabetes in a 14-Year Follow-Up: Lessons from the San Luigi Gonzaga Diabetes Study. Diabetes Care 34, 2237-2243.
Le Chatelier, E., Nielsen, T., Qin, J., Prifti, E., Hildebrand, F., Falony, G., Almeida, M., Arumugam, M., Batto, J.-M., Kennedy, S., et al. (2013). Richness of human gut microbiome correlates with metabolic markers. Nature 500, 541-546.
Conn, J. W., and Newburgh, L. H. (1936). The glycemic response to isoglucogenic quantities of protein and carbohydrate. J. Clin. Invest. 15, 665-671.
Cunningham, K. M., and Read, N. W. (1989). The effect of incorporating fat into different components of a meal on gastric emptying and postprandial blood glucose and insulin responses. Br. J. Nutr. 61, 285-290.
David, L. A., Maurice, C. F., Carmody, R. N., Gootenberg, D. B., Button, J. E., Wolfe, B. E., Ling, A. V, Devlin, A. S., Varma, Y., Fischbach, M. A., et al. (2014). Diet rapidly and reproducibly alters the human gut microbiome. Nature 505, 559-563.
Dodd, H., Williams, S., Brown, R., and Venn, B. (2011). Calculating meal glycemic index by using measured and published food values compared with directly measured meal glycemic index. Am. J. Clin. Nutr. 94, 992-996.
Duncan, S. H., Belenguer, A., Holtrop, G., Johnstone, A. M., Flint, H. J., and Lobley, G. E. (2007). Reduced dietary intake of carbohydrates by obese subjects results in decreased concentrations of butyrate and butyrate-producing bacteria in feces. Appl. Environ. Microbiol. 73, 1073-1078.
Dunstan, D. W., Kingwell, B. A., Larsen, R., Healy, G. N., Cerin, E., Hamilton, M. T., Shaw, J. E., Bertovic, D. A., Zimmet, P. Z., Salmon, J., et al. (2012). Breaking up prolonged sitting reduces postprandial glucose and insulin responses. Diabetes Care 35, 976-983.
Edgar, R. C. (2013). UPARSE: highly accurate OTU sequences from microbial amplicon reads. Nat. Methods 10, 996-998.
Everard, A., Lazarevic, V., Derrien, M., Girard, M., Muccioli, G. G., Muccioli, G. M., Neyrinck, A. M., Possemiers, S., Van Holle, A., Francois, P., et al. (2011). Responses of gut microbiota and glucose and lipid metabolism to prebiotics in genetic obese and diet-induced leptin-resistant mice. Diabetes 60, 2775-2786.
Everard, A., Lazarevic, V., Gala, N., Johansson, M., Stahlman, M., Backhed, F., Delzenne, N. M., Schrenzel, J., Francois, P., and Cani, P. D. (2014). Microbiome of prebiotic-treated mice reveals novel targets involved in host response during obesity. ISME J. 8, 2116-2130.
Foster-Powell, K., Holt, S. H. A., and Brand-Miller, J. C. (2002). International table of glycemic index and glycemic load values: 2002. Am. J. Clin. Nutr. 76, 5-56.
Friedman, J. H. (2001). Greedy Function Approximation: A Gradient Boosting Machine. Ann. Stat. 29, 1189-1232.
Gallwitz, B. (2009). Implications of postprandial glucose and weight control in people with type 2 diabetes: understanding and implementing the International Diabetes Federation guidelines. Diabetes Care 32 Suppl 2, S322-S325.
Gibbs, E. M., Stock, J. L., McCoid, S. C., Stukenbrok, H. A., Pessin, J. E., Stevenson, R. W., Milici, A. J., and McNeish, J. D. (1995). Glycemic improvement in diabetic db/db mice by overexpression of the human insulin-regulatable glucose transporter (GLUT4). J. Clin. Invest. 95, 1512-1518.
Greenwood, D. C., Threapleton, D. E., Evans, C. E. L., Cleghorn, C. L., Nykjaer, C., Woodhead, C., and Burley, V. J. (2013). Glycemic Index, Glycemic Load, Carbohydrates, and Type 2 Diabetes: Systematic review and dose-response meta-analysis of prospective studies. Diabetes Care 36, 4166-4171.
Grundy, S. M. (2012). Pre-diabetes, metabolic syndrome, and cardiovascular risk. J. Am. Coll. Cardiol. 59, 635-643.
Hastie, T., Tibshirani, R., and Friedman, J. (2008). The Elements of Statistical Learning: Data Mining, Inference and Prediction (Stanford: Springer).
Himsworth, H. P. (1934). Dietetic factors influencing the glucose tolerance and the activity of insulin. J. Physiol. 81, 29-48.
Israeli Center for Disease Control (2014). Health 2013 (Jerusalem: Israeli Ministry of Health).
Jenkins, D. J., Wolever, T. M., Taylor, R. H., Barker, H., Fielden, H., Baldwin, J. M., Bowling, A. C., Newman, H. C., Jenkins, A. L., and Goff, D. V (1981). Glycemic index of foods: a physiological basis for carbohydrate exchange. Am. J. Clin. Nutr. 34, 362-366.
Kanehisa, M., and Goto, S. (2000). KEGG: kyoto encyclopedia of genes and genomes. Nucleic Acids Res. 28, 27-30.
Karlsson, F. H., Tremaroli, V., Nookaew, I., Bergstrom, G., Behre, C. J., Fagerberg, B., Nielsen, J., and Bäckhed, F. (2013). Gut metagenome in European women with normal, impaired and diabetic glucose control. Nature 498, 99-103.
Korem, T., Zeevi, D., Suez, J., Weinberger, A., Avnit-Sagi, T., Pompan-Lotan, M., Matot, E., Jona, G., Harmelin, A., Cohen, N., et al. (2015). Growth dynamics of gut microbiota in health and disease inferred from single metagenomic samples. Science 349, 1101-1106.
Kristo, A. S., Matthan, N. R., and Lichtenstein, A. H. (2013). Effect of diets differing in glycemic index and glycemic load on cardiovascular risk factors: review of randomized controlled-feeding trials. Nutrients 5, 1071-1080.
Lamkin, D. M., Spitz, D. R., Shahzad, M. M. K., Zimmerman, B., Lenihan, D. J., Degeest, K., Lubaroff, D. M., Shinn, E. H., Sood, A. K., and Lutgendorf, S. K. (2009). Glucose as a prognostic factor in ovarian carcinoma. Cancer 115, 1021-1027.
Li, J., Jia, H., Cai, X., Zhong, H., Feng, Q., Sunagawa, S., Arumugam, M., Kultima, J. R., Prifti, E., Nielsen, T., et al. (2014). An integrated catalog of reference genes in the human gut microbiome. Nat. Biotechnol. 32, 834-841.
Marco-Sola, S., Sammeth, M., Guigó, R., and Ribeca, P. (2012). The GEM mapper: fast, accurate and versatile alignment by filtration. Nat. Methods 9, 1185-1188.
Martínez, I., Lattimer, J. M., Hubach, K. L., Case, J. A., Yang, J., Weber, C. G., Louk, J. A., Rose, D. J., Kyureghian, G., Peterson, D. A., et al. (2013). Gut microbiome composition is linked to whole grain-induced immunological improvements. ISME J. 7, 269-280.
Nathan, D. M., Davidson, M. B., DeFronzo, R. A., Heine, R. J., Henry, R. R., Pratley, R., and Zinman, B. (2007). Impaired fasting glucose and impaired glucose tolerance: implications for care. Diabetes Care 30, 753-759.
Nielsen, H. B., Almeida, M., Juncker, A. S., Rasmussen, S., Li, J., Sunagawa, S., Plichta, D. R., Gautier, L., Pedersen, A. G., Le Chatelier, E., et al. (2014). Identification and assembly of genomes and genetic elements in complex metagenomic samples without using reference genomes. Nat. Biotechnol. 32, 822-828.
Nishida, T., Tsuji, S., Tsujii, M., Arimitsu, S., Haruna, Y., Imano, E., Suzuki, M., Kanda, T., Kawano, S., Hiramatsu, N., et al. (2006). Oral Glucose Tolerance Test Predicts Prognosis of Patients with Liver Cirrhosis. Am. J. Gastroenterol. 101, 70-75.
Qin, J., Li, Y., Cai, Z., Li, S., Zhu, J., Zhang, F., Liang, S., Zhang, W., Guan, Y., Shen, D., et al. (2012). A metagenome-wide association study of gut microbiota in type 2 diabetes. Nature 490, 55-60.
Riccardi, G., and Rivellese, A. A. (2000). Dietary treatment of the metabolic syndrome—the optimal diet. Br. J. Nutr. 83 Suppl 1, S143-S148.
Ridaura, V. K., Faith, J. J., Rey, F. E., Cheng, J., Duncan, A. E., Kau, A. L., Griffin, N. W., Lombard, V., Henrissat, B., Bain, J. R., et al. (2013). Gut microbiota from twins discordant for obesity modulate metabolism in mice. Science 341, 1241214.
Sandkvist, M. (2001). Type II secretion and pathogenesis. Infect. Immun. 69, 3523-3535.
Santacruz, A., Marcos, A., Wärnberg, J., Martí, A., Martin-Matillas, M., Campoy, C., Moreno, L. A., Veiga, O., Redondo-Figuero, C., Garagorri, J. M., et al. (2009). Interplay Between Weight Loss and Gut Microbiota Composition in Overweight Adolescents. Obesity 17, 1906-1915.
Schwingshackl, L., and Hoffmann, G. (2013). Long-term effects of low glycemic index/load vs. high glycemic index/load diets on parameters of obesity and obesity-associated risks: A systematic review and meta-analysis. Nutr. Metab. Cardiovasc. Dis. 23, 699-706.
Suez, J., Korem, T., Zeevi, D., Zilberman-Schapira, G., Thaiss, C. A., Maza, O., Israeli, D., Zmora, N., Gilad, S., Weinberger, A., et al. (2014). Artificial sweeteners induce glucose intolerance by altering the gut microbiota. Nature 514, 181-186.
The Human Microbiome Project Consortium (2012). Structure, function and diversity of the healthy human microbiome. Nature 486, 207-214.
Truong, D. T., Franzosa, E. A., Tickle, T. L., Scholz, M., Weingart, G., Pasolli, E., Tett, A., Huttenhower, C., and Segata, N. (2015). MetaPhlAn2 for enhanced metagenomic taxonomic profiling. Nat. Methods 12, 902-903.
Turnbaugh, P. J., Ley, R. E., Mahowald, M. A., Magrini, V., Mardis, E. R., and Gordon, J. I. (2006). An obesity-associated gut microbiome with increased capacity for energy harvest. Nature 444, 1027-1031.
Turnbaugh, P. J., Hamady, M., Yatsunenko, T., Cantarel, B. L., Duncan, A., Ley, R. E., Sogin, M. L., Jones, W. J., Roe, B. A., Affourtit, J. P., et al. (2009). A core gut microbiome in obese and lean twins. Nature 457, 480-484.
Vega-Lopez, S., Griffith, J. L., Ausman, L. M., and Lichtenstein, A. H. (2007). Interindividual Variability and Intra-Individual Reproducibility of Glycemic Index Values for Commercial White Bread. Diabetes Care 30, 1412-1417.
Vrolix, R., and Mensink, R. P. (2010). Variability of the glycemic response to single food products in healthy subjects. Contemp. Clin. Trials 31, 5-11.
Wolever, T. M., and Jenkins, D. J. (1986). The use of the glycemic index in predicting the blood glucose response to mixed meals. Am. J. Clin. Nutr. 43, 167-172.
World Health Organization (2008). Global Health Observatory Data Repository, Disease and Injury Country Estimates.
Wu, G. D., Chen, J., Hoffmann, C., Bittinger, K., Chen, Y.-Y., Keilbaugh, S. a, Bewtra, M., Knights, D., Walters, W. a, Knight, R., et al. (2011). Linking long-term dietary patterns with gut microbial enterotypes. Science 334, 105-108.
Xiao, S., Fei, N., Pang, X., Shen, J., Wang, L., Zhang, B., Zhang, M., Zhang, X., Zhang, C., Li, M., et al. (2014). A gut microbiota-targeted dietary intervention for amelioration of chronic inflammation underlying metabolic syndrome. FEMS Microbiol. Ecol. 87, 357-367.
Zhang, X., Shen, D., Fang, Z., Jie, Z., Qiu, X., Zhang, C., Chen, Y., and Ji, L. (2013). Human gut microbiota changes reveal the progression of glucose intolerance. PLoS One 8, e71108.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of preventing diabetes or pre-diabetes in a subject comprising administering to the subject at least one bacteria of a phylum, class, order, family, genus or species of a bacteria which is categorized as beneficial according to Table 3, Table 4, or Table 5, thereby preventing diabetes or prediabetes in the subject.

2-4. (canceled)

5. A probiotic composition, comprising at least two bacteria of a phylum, class, order, family, genus or species of a bacteria which is categorized as beneficial according to Table 3, Table 4 or Table 5.

6-20. (canceled)

21. A method of improving the glucose response in a glucose intolerant subject comprising providing to the subject a probiotic composition comprising at least one bacteria species selected from the group consisting of Coprococcus sp. ART55/1 draft, vButyrate-producing bacterium SSC/2, Roseburia intestinalis XB6B4 draft, Eubacterium siraeum V10Sc8a draft, Veillonella parvula DSM 2008 chromosome, Ruminococcus sp. SR1/5 draft, Ruminococcus bromii L2-63 draft, Bacteroides thetaiotaomicron VPI-5482 chromosome, Faecalibacterium prausnitzii L2-6, Bifidobacterium adolescentis ATCC 15703 chromosome, Ruminococcus obeum A2-162 draft, Bacteroides xylanisolvens XB1A draft, Treponema succinifaciens DSM 2489 chromosome, Bacteroides vulgatus ATCC 8482 chromosome, Klebsiella pneumoniae subsp. pneumoniae HS11286 chromosome, Eubacterium siraeum 70/3 draft, Bifidobacterium bifidum BGN4 chromosome, Methanobrevibacter smithii ATCC 35061 chromosome, Eubacterium eligens ATCC 27750 chromosome, Eubacterium rectale M104/1 draft, Megamonas hypermegale ART12/1 draft, Lactobacillus ruminis ATCC 27782 chromosome, Escherichia coli SE15, Streptococcus pyogenes MGAS2096 chromosome, Bifidobacterium longum subsp. longum F8 draft, Klebsiella pneumoniae JM45, Escherichia coli str. ‘clone D i2’ chromosome, Klebsiella oxytoca KCTC 1686 chromosome, Raoultella ornithinolytica B6, Methylocella silvestris, Roseiflexus castenholzii and Streptococcus macedonicus, wherein the probiotic composition does not comprise more than 50 species of bacteria, thereby improving the glucose response in a glucose intolerant subject.

22. (canceled)

23. The method of claim 21, wherein said glucose intolerant subject is a diabetic subject or a prediabetic subject.

24-38. (canceled)

39. The method of claim 1, wherein the bacteria is categorized as being beneficial according to Table 3.

40. The method of claim 1, wherein the bacteria is categorized as being beneficial according to Table 4.

41. The method of claim 1, wherein the bacteria is categorized as being beneficial according to Table 5.

42. The probiotic composition of claim 5, wherein the bacteria is categorized as being beneficial according to Table 3.

43. The probiotic composition of claim 5, wherein the bacteria is categorized as being beneficial according to Table 4.

44. The probiotic composition of claim 5, wherein the bacteria is categorized as being beneficial according to Table 5.

45. The probiotic composition of claim 5, not comprising more than 50 species of bacteria.

46. The probiotic composition of claim 5, being formulated for rectal or oral administration.

47. The probiotic composition of claim 21, being formulated for rectal or oral administration.
